SEQUENCE LISTING (61 sequences)

SEQ ID NO: 1 - DNA, 5444 bp, Artificial Sequence
  Nucleotide sequence of Cas9, including a C-terminal SV40 nuclear localization signal, codon pair optimized for expression in Yarrowia lipolytica
gtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattatta

SEQ ID NO: 2 - DNA, 11606 bp, Artificial Sequence
  Nucleotide sequence of vector MB7452
cgcgtggatcgccggtgcgttgacgttggtgacctccagccagaggtgcccggcgccccgctcgccggcgaactccgtcgcgagccccatcaacgcgcgcccgaccccgtgcccccggtgctccggggcgacctcgatgtcctcgacggtcagccggcggttccacgccgagtacgagatgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgccgctcgccgtccgaggcggtgccgtcgtacagggcggtgtccaggccgcagagggtgaaccccatccgccggta

SEQ ID NO: 3 - DNA, 12810 bp, Artificial Sequence
  Nucleotide sequence of vector pSTV086
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgaggccactgatgagtccgtgaggacgaaacgagtaagctcgtctggcctgttgagtcaacccggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 4 - DNA, 2142 bp, Artificial Sequence
  Nucleotide sequence of the cassette of the donor DNA for expression of GFP (Yl_HSP.pro - A.vic_eGFP ORF - Yl_GPD.ter)
tgtgaccttgtaaacaacaacccgactttacagtaacgcttggacacactgtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactacaatggagggtgatattcacggtgcttctaagggtgaggagcttttcaccggcgttgtccctattctcgttgagcttgatggtgatgtcaacggtcacaagttttctgtctctggtgagggtgagggtgatgctacttacggtaagctcaccctcaagtttatttgtaccaccggcaagctccccgttccctggcctaccctcgttaccactctcacctacggtgttcagtgtttttcccgataccccgatcacatgaagcgacacgactttttcaagtctgccatgcccgagggttacgttcaggagcgaactatttctttcaaggatgacggtaactacaagacccgagctgaggttaagtttgagggtgataccctcgttaaccgaatcgagcttaagggtattgatttcaaggaggatggtaacattctcggtcacaagctggagtacaactacaactctcacaacgtttacatcaccgctgacaagcagaagaacggtatcaaggctaacttcaagattcgacacaacattgaggatggttccgttcagcttgctgaccactaccagcagaacactcccattggcgatggccctgtcctcctccccgacaaccactacctctctacccagtctgccctttctaaggaccccaacgagaagcgagatcacatggtcctccttgagttcgttaccgctgctggtattactcacggtatggatgagctctacaagtaaataaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacatcaatatatcggaccaatatatcggaccacggtcgccgccgaatcgcc

SEQ ID NO: 5 - DNA, 1075 bp, Artificial Sequence
  Nucleotide sequence of the 5′ bipartite donor DNA fragment, comprising the 50 bp genomic sequence of INT05, the Yl_HSP.promoter and part of the A.vic_eGFP ORF
tgtgaccttgtaaacaacaacccgactttacagtaacgcttggacacactgtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactacaatggagggtgatattcacggtgctt

SEQ ID NO: 6 - DNA, 1117 bp, Artificial Sequence
  Nucleotide sequence of the 3′ bipartite donor DNA fragment, comprising part of the Yl_HSP promoter - A.vic_eGFP ORF - Yl.GPD terminator and 50 bp genomic sequence of INT05
caccaacacacacacttatactacaatggagggtgatattcacggtgcttctaagggtgaggagcttttcaccggcgttgtccctattctcgttgagcttgatggtgatgtcaacggtcacaagttttctgtctctggtgagggtgagggtgatgctacttacggtaagctcaccctcaagtttatttgtaccaccggcaagctccccgttccctggcctaccctcgttaccactctcacctacggtgttcagtgtttttcccgataccccgatcacatgaagcgacacgactttttcaagtctgccatgcccgagggttacgttcaggagcgaactatttctttcaaggatgacggtaactacaagacccgagctgaggttaagtttgagggtgataccctcgttaaccgaatcgagcttaagggtattgatttcaaggaggatggtaacattctcggtcacaagctggagtacaactacaactctcacaacgtttacatcaccgctgacaagcagaagaacggtatcaaggctaacttcaagattcgacacaacattgaggatggttccgttcagcttgctgaccactaccagcagaacactcccattggcgatggccctgtcctcctccccgacaaccactacctctctacccagtctgccctttctaaggaccccaacgagaagcgagatcacatggtcctccttgagttcgttaccgctgctggtattactcacggtatggatgagctctacaagtaaataaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacatcaatatatcggaccaatatatcggaccacggtcgccgccgaatcgcc

SEQ ID NO: 7 - DNA, 20 bp, Artificial Sequence
  Nucleotide sequence of the genomic target of integration locus INT05 in the Yarrowia lipolytica genome
tggcctgttgagtcaacccg

SEQ ID NO: 8 - DNA, 2515 bp, Artificial Sequence
  Nucleotide sequence of the integration locus INT05
tttaatagatgtttgtgttggttggttggttggttggtcgattggttgttcgtgtcagatgtgtagcttggaggcttgatactgaaactatcgagcgtgtggagaggtctttatactagccacaccatgtcatgctcgtatacatgttttagacttctcgctaaccttacgcccccgctgaccttttccggcactgtgattggccggttggtcccataacgcacgtgacccatcagtggcgtgtctcgctgacctttgctggggatttgattggctgacgaggagggtgaatcacgtgatgatctgagaggtgcacagatatgctgacctttgaagcgcgtctgattggctgggtcgtgaccaagtcacgtgatcccgagagtggaacattcccacaacctgtttaatcatggcgaatacacagggctaaggtggctggtaaggtggccaaaataagacgcaaaaagtgaggcttgtgtgtgggttatcagattgtctctggaggggcgaatacgttgggaaaaaaatggaaaaacgggaggaattgctgagtaatatttatttggaccttgtgaccgacccgtgtcgcagcaaacataatcctggttttgatggacgatgccagtatcgagtatgtagaagaacagtttcaagtaagagacgtggtgtccatagagctgccgatgatactcactgtaaaggtacatactacagtagtccatagtcttgaacattcaatgcgctccaatgttgtgatgaatattgcgtcttaccatgaagagtggttgcagaagttgtatcatttgattggacatttcacgctgatttgattcgtatggcaaaatctttttccttacagaacgtcgactttttgtagttgtgctgtggtaaaataatcggacaattttggccgaaagtggcagacttttgacgaggtttgatcgacgatctatgaaaattttaatcaaaatcataattaaaaaataaataaataaataaatcaaataaaaatcaaataaaaatcaaataaaaatcaaataaaaaaatcaaatatataaataaaaaaaaaaaaataaaaaaccattgatcttactgtcgaatgtacgatacagcaccactcgcactgtcttatatgtacgtatgctgtactctcgtatcagcctattcccagaagtgataaaaaagaaccgtcgctgttgatgttgacgaaaacagtccaacatcctattcgtacagtgcatacgactacggtacacgaacttgtatctctcctgttccgctgttggtcgcaccatcgcttaccgtctagttggttaaacctattgtatgtactgtacatactcgtacttgtaactaataaccaccgttatccacgccgttatccgttgtgccattgtttacttgtatcgtacttcggctaatcgcctgagtggtgtgagtataaaaaatggcagtcacacttgtattgctgtactgcgccacaccttagctgaccatacctatgacgttccacgacgagcaccggtccggcacccgactcgtactcgtcgtacttgcagcctcatccatacccagcaatgatgccatgttcctatccagggcacggcctcttcatcacaccacctgtgcttccgagactatatgtaagcaagttgggcagtgtaagtggtgcaggtcatgtgaccttgtaaacaacaacccgactttacagtaacgcttggacacacttgttggcctgttgagtcaacccggggccaacccaaggcaacaaggagaccaaaccatcaatatatcggaccaatatatcggaccacggtcgccgccgaatcgccccacatggcgctgggccgttccagctgagctgatgccccccacgtcgctcccatcgtatatccaaaccctatcagcattttttctgaaaaaggccgttaaacgacatatacgacttgggagttggatcagtaggctgatgttgatttttcaacccattttaccaaaccaaaaagctaatctagccgagtcgatggcggatcgcggacatgctactctagaccacgttccggacggcggtttcgaccggcgaggttatgagtacgtcagaagcagacagaagaacagattgcgagcacaaatccgagacccacggccccctaaaacacgtccagggccctaatcagatgtgccgtcgcgacgcccctgacgtgttcacatgttgagtttggatgcagaaggttcagcatgtttagcccgtttgggttatcagatgattgcttgcgggggtcgttggggaccatttccacacgaatacacggaaaaaaaccacacagtcacactcaccgtgtcagtgccatcatgttggtgccctttaccccccaaatatcccagtttacacatcttgcaacccccccgggatacgacgcaaacgacagggttaggagcaaaggctaatacttttcgtgttttataggccctcccatcccaccctttggagtcccatatctcacctcgacacacc

SEQ ID NO: 9 - DNA, 6 bp, Artificial Sequence
  Nucleotide sequence of 6 bp inverted repeat of the INT05 genomic target
aggcca

SEQ ID NO: 10 - DNA, 37 bp, Artificial Sequence
  Nucleotide sequence of HH ribozyme
ctgatgagtccgtgaggacgaaacgagtaagctcgtc

SEQ ID NO: 11 - DNA, 148 bp, Artificial Sequence
  Nucleotide sequence of HDV ribozyme
gttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggac

SEQ ID NO: 12 - DNA, 973 bp, Artificial Sequence
  Nucleotide sequence of Yl_HYPO promoter
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatg

SEQ ID NO: 13 - DNA, 300 bp, Artificial Sequence
  Nucleotide sequence of Yl_GPD terminator
atatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattatta

SEQ ID NO: 14 - DNA, 303 bp, Artificial Sequence
  Nucleotide sequence of the Yl_PGM terminator
taaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataata

SEQ ID NO: 15 - DNA, 1004 bp, Artificial Sequence
  Nucleotide sequence of the Yl_007 promoter
gtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaaca

SEQ ID NO: 16 - DNA, 1000 bp, Artificial Sequence
  Nucleotide sequence of the Yl_HSP promoter
gtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactaca

SEQ ID NO: 17 - DNA, 22 bp, Artificial Sequence
  Nucleotide sequence of the forward primer for amplification of donor DNA fragment INT05 5′ FLANK - Yl_HSP.pro - A.vic_eGFP ORF - Yl_GPD.ter - INT05 3′ FLANK
tgtgaccttgtaaacaacaacc

SEQ ID NO: 18 - DNA, 18 bp, Artificial Sequence
  Nucleotide sequence of the reverse primer for amplification of donor DNA fragment INT05 5′ FLANK - Yl_HSP.pro - A.vic_eGFP ORF - Yl_GPD.ter - INT05 3′ FLANK
ggcgattcggcggcgacc

SEQ ID NO: 19 - DNA, 22 bp, Artificial Sequence
  Nucleotide sequence of the forward primer for amplification of the 5′ bipartite donor DNA fragment
tgtgaccttgtaaacaacaacc

SEQ ID NO: 20 - DNA, 20 bp, Artificial Sequence
  Nucleotide sequence of the reverse primer for amplification of the 5′ bipartite donor DNA fragment
aagcaccgtgaatatcaccc

SEQ ID NO: 21 - DNA, 21 bp, Artificial Sequence
  Nucleotide sequence of the forward primer for amplification of the 3′ bipartite donor DNA fragment
caccaacacacacacttatac

SEQ ID NO: 22 - DNA, 18 bp, Artificial Sequence
  Nucleotide sequence of the reverse primer for amplification of the 3′ bipartite donor DNA fragment
ggcgattcggcggcgacc

SEQ ID NO: 23 - DNA, 2192 bp, Artificial Sequence
  Nucleotide sequence of the donor DNA fragment which comprises the crtE expression cassette
tcagccgacggactcagcggcctaatggacttcagaacacttcactccacaacgttgtccaggtttgtatccacgtgtgtccgttccgccaatattccgcgtgctcttgcgctttctacgcagtttccattctctttctatgcacatctcggcaagtgtctcacatctttccttttcgcccacaaggactcacctgtacaagtatcagtacagcacgttgtgcaagcttcattagacagcaggcttaagtcatagacagtgtcgtattgctgttggattactggcagttaagcacttatttagactgacatttatactgtattctgctgagtctgagacagcgagctcatgtgtctttccatagcacagctacttatacagtagcaaatgtatcggaagtggcaaattggagcaagcgccagttattcgacattttttaatcagtggaggctttctataaaaaatgaaccatgcattttctcttgaaattcgttttgaaacccttgtcaatacaatagtacaagtacccatccagtccctctcaacaagtctagccgactcgtttccactgtgcctcctacacgcacatattgcgttatcttctaatgggttctctgtggaaccctaatccaccccgcaccccacaatgccatatatagaggagggcgcaatgtacttgttcctcaacacaacacgactacaaaatggactacgccaacattctgaccgccatccctctcgagttcactccccaggacgacattgttctgctcgagccctaccactacctcggtaagaaccccggcaaggagatccgatcccagctcattgaggccttcaactactggctcgatgtcaagaaagaggatctcgaggtcatccagaacgttgttggtatgctccacaccgcctctctgctcatggacgatgtcgaggactcctccgttctgcgacgaggctcccccgttgcccacctcatctacggtatcccccagaccatcaacaccgccaactacgtctacttcctcgcctaccaggagatcttcaagctccgacccacccccatccccatgcccgtcattcctccctcctctgcttctctgcagtcctccgtctcctccgcttcttcctcctcttctgcttcttccgagaacggtggtacctccactcccaactcccagatccccttctccaaggacacctacctcgacaaggtcatcaccgacgagatgctgtctctgcaccgaggccagggtctggagctgttctggcgagactctctgacctgcccctccgaggaagagtacgtcaagatggttctcggcaagaccggtggcctcttccgaattgccgtccgactcatgatggccaagtccgagtgcgacattgactttgtccagctcgtcaacctcatctccatctacttccagatccgagacgactacatgaacctccagtcttccgagtacgcccacaacaagaactttgctgaggacctcaccgagggtaagttctccttccccaccatccactccatccacgccaacccctcttctcgactcgtcatcaacactctgcagaagaagtccacctcccccgagattctgcaccactgtgtcaactacatgcgaaccgagactcactccttcgagtacacccaggaagttctcaacaccctctccggtgctctcgagcgagagctcggccgactccagggtgagtttgccgaggccaactccaagattgacctcggtgatgtcgagtccgagggccgaaccggcaagaacgtcaagctcgaggccatcctcaagaagctcgctgacatccccctttaataaattacttttagttagctaatgtatgtatgattttgaccaggaactacgaatgcaaagcacaagttctacaagtagacgtgttgtatacctgataccatgttcttgtgtactcatgatcgtggacgtgttgttacatgttatatcaagcgtcatgattgcgactcataatggattgtgcgttattttgggtgaggtaattccaacaagtgttgggcaagcaattgtacgatagatcaagccatgactctacagtataatgaaaggagcgtgtgctcgttgtaggtactcaggtgaaataatcctcaaataaccacaaacatccttcccatatgctcggtcgtgcttgttgtacct

SEQ ID NO: 24 - DNA, 3033 bp, Artificial Sequence
  Nucleotide sequence of the donor DNA fragment which comprises the crtYB expression cassette
aaataaccacaaacatccttcccatatgctcggtcgtgcttgttgtacctgtgcgaatcgaaagagccactgtttccctgctccctcaaaatatgcaattacaaatcatcgccatctacagacgcatatggacacatgcacatacttgcactccacagcaactcatgcgagtcttgcatgagtggcaaatggcagtggcgttagcaagagacgagatgcggcgtagataatcgatttgatgcacaccaattgtaacgttggcggtgaagcctatttggcaacccgccccgccacatggcattgtcgtaggctgggatttttagtccgaaattcgaggtggttttttgaagtgagacttgtttcttggaagtttaatgttttgacggggatgagtggaaagtggatggaaatttgaggtggtagaggtcggtcaattggtgagatttttgaataaatcaattctccagggaatttcgagctgattgccattttccgcatctttttacgacctcaaaacctcgagataattgcgtcagaaaaagaaaaacaatacctcgccacatgccaattaaccatcagataaccatatttaaaaagtatatataggtagtccagttcccacccctctgccaggtccccccaaccaccatcaaaatgaccgccctcgcctactaccagatccacctcatctacaccctccccatcctcggcctcctcggtctgctcacctcccccattctgaccaagttcgacatctacaagatctccatcctcgtcttcattgccttctccgccaccactccctgggactcctggatcatccgaaacggtgcctggacctacccctctgctgagtccggccagggtgtctttggtaccttcctcgatgtcccctacgaggagtacgctttctttgtcatccagaccgtcatcactggtctggtctacgttctcgccacccgacacctgctcccctctctcgccctccccaagacccgatcttccgctctgtctctggctctcaaggctctcatccccctgcccatcatctacctcttcaccgcccacccttccccctcccccgaccccctcgtcaccgaccactacttctacatgcgagctctgtccctcctcatcactccccctaccatgctcctcgctgctctgtccggtgagtacgcctttgactggaagtccggccgagccaagtccaccattgctgccatcatgatccccactgtctacctcatctgggtcgactacgttgccgtcggccaggactcttggtccatcaacgacgagaagattgtcggctggcgactcggcggtgttctccccatcgaagaggccatgttcttcctgctcaccaacctcatgattgttctcggcctctccgcctgcgaccacacccaggctctctacctcctccacggccgaaccatctacggcaacaagaagatgccctcttctttccccctcatcaccccacccgttctgtctctcttcttctcctctcgaccctactcctcccagcccaagcgagatctggagctcgccgtcaagctcctcgagaagaagtcccgatctttcttcgttgcctccgctggtttcccctccgaggtccgagagcgactcgtcggcctctacgccttctgccgagtcaccgatgatctcattgactcccccgaggtttcctccaacccccacgccaccattgacatggtttccgacttcctcaccctcctcttcggtcctcccctgcacccctctcagcccgacaagattctctcctctcccctgctgcctccctcccacccctcccgacccaccggtatgtaccccctgcctcccccgccctctctgtcccccgccgagctggtccagttcctcaccgagcgagtccccgtccagtaccactttgccttccgactgctggccaagctccagggtctgatcccccgataccccctcgacgagctcctgcgaggctacaccaccgacctcatcttccccctgtccaccgaggccgtccaggcccgaaagacccccattgagactactgctgacctcctcgactacggtctgtgtgttgccggctccgtcgccgagctcctcgtctacgtctcttgggcctctgctccctctcaggtccccgccactattgaggagcgagaggctgttctggttgcctcccgagagatgggtaccgccctccagctcgtcaacattgcccgagacatcaagggtgacgccaccgagggtcgattctacctccccctctccttcttcggtctgcgagacgagtccaagctcgccatccccaccgactggaccgagccccgacctcaggactttgacaagctgctctccctctccccctcctctaccctcccctcctccaacgcctccgagtccttccgatttgagtggaagacctactctctgcccctcgttgcttacgccgaggatctcgccaagcactcctacaagggtatcgaccgactccccactgaggttcaggccggtatgcgagctgcctgcgcctcttacctcctcattggccgagagatcaaggtcgtctggaagggtgatgtcggtgagcgacgaaccgttgccggttggcgacgagtccgaaaggtcctctccgtcgtcatgtccggttgggagggccagtaataaacaagcgggtatttattgtatgaataaagattatgtattgattgcaaaaaagtgcatttgtagatgtggtttattgtagagagtacggtatgtactgtacgaacattaggagctacttctacaagtagattttcttaacaagggtgaaatttactaggaagtacatgcatatttcgttagtagaatcacaaaagaaatgtacaagcacgtactacttgtactccacaatgtggagtgggagcaaaaaaattggacgacaccggaatcgaaccggggacctcgcgcatgctaagcgcagcacctcgaaaccttcgaatccagccagcatgtcgacacccacaagatgtagtgcac

SEQ ID NO: 25 - DNA, 3210 bp, Artificial Sequence
  Nucleotide sequence of the donor DNA fragment which comprises the crtI expression cassette
gaaaccttcgaatccagccagcatgtcgacacccacaagatgtagtgcacgtgcccttttgccagtatatccaccgcagcacccaccatgagcgacatctgatatcgtgccgcgaccactaccccaaataagctccaactaatatgccgaggcaggtgggaaactatgcactccagttgacgctgtagaagcacatggaaggtgcggaggcggtggcaacgaggggcatgagccatcaacgaggaaccacagacaaggcaaggggggaaacgcgaccggaatctctcgcggtcacgtgacccgcccgggttcactcgtccatgttgtgtctctggtgtcttcggccgactcgcattggttaaacttccaccaccgcaatcacgtcccactggccaaactttttctgctttctctgactttttctggccaaaaggcaacgtcggaaagggtcgggaggattcggaaccgacgaaaatcggccggctccagcgggggtagttcggcagtcctggtgggagcgctaggggagctgtggtctgtgtagggcgcgggtccgggtttgttgggtgtcaaatcacgtgtttttgcccccccgctgagccggactccgacaaccgtgtctccaacggcctgactaagctgctcccagcactctgccgtagcgttggtctgtcctgtcgcactctgttcaaagacagaagaaagaaaaagctaacctccacgtcagagacaatggtagaaggcttgttccttgcaaccgaggagagtgagtgttctcggcacgagcatcatgggcgatctggagggtatttttgaggggaaaaaacgggatcaggacaaacagaggccacagaccgggaatctgggccccaaaacggccttttcccgtcgcaaaaccggtctacatacaccccttcggcccgccacaggccggtgtgaaaaaccctaaagcttgcttcaaaccagacggacgcacagcaagacacatcatgaagagtcacctgcagtatatatagatctggggatccccagtagactgaccaagcatacaaaaatgggcaaggagcaggaccaggacaagcctaccgccatcattgtcggctgtggtatcggtggtattgccactgctgctcgactcgccaaggaaggcttccaggtcaccgtctttgagaagaacgactactccggcggccgatgctctctcattgagcgagacggctaccgattcgaccagggcccctccctgctcctcctccccgatctcttcaagcagacctttgaggatctcggtgagaagatggaggactgggttgacctcatcaagtgtgagcccaactacgtctgccacttccacgacgaggagactttcaccttctccaccgacatggctctgctcaagcgagaggtcgagcgattcgagggcaaggacggctttgaccgattcctgtctttcatccaggaagcccaccgacactacgagctcgccgttgtccacgttctccagaagaacttccccggctttgccgccttcctgcgactccagttcatcggtcagatcctcgctctgcaccccttcgagtccatctggacccgagtctgccgatacttcaagaccgaccgactccgacgagtcttctccttcgccgttatgtacatgggccagtccccctactccgctcccggtacctactctctgctccagtacaccgagctcaccgagggtatctggtacccccgaggtggtttctggcaggtccccaacaccctcctccagatcgtcaagcgaaacaacccctccgccaagttcaacttcaacgcccccgtctcccaggttctcctctcccccgccaaggaccgagccaccggtgtccgacttgagtccggtgaggagcaccacgccgatgttgtcattgtcaacgccgacctcgtctacgcctccgagcacctcatccccgatgatgcccgaaacaagatcggtcagctcggtgaggtcaagcgatcttggtgggccgatctggtcggtggcaagaagctgaagggctcttgctcctctctgtccttctactggtctatggaccgaatcgtcgacggcctcggtggccacaacatcttcctggccgaggacttcaagggctccttcgacaccatcttcgaggagctcggcctccccgctgacccctccttctacgtcaacgtcccctctcgaatcgacccctctgctgctcctgagggtaaggacgccattgtcatcctcgtcccctgcggtcacattgatgcctccaacccccaggactacaacaagctcgttgcccgagcccgaaagtttgtcatccagaccctctccgctaagctcggtctgcccgacttcgagaagatgattgttgccgagaaggtccacgacgccccctcttgggagaaggagttcaacctcaaggacggttccatcctcggtctggcccacaacttcatgcaggttctcggcttccgaccctccacccgacaccccaagtacgacaagctgttctttgtcggtgcctccacccaccccggtactggtgtccccattgttctggctggtgccaagctcactgccaaccaggttctggagtccttcgaccgatctcctgctcccgaccccaacatgtctctctccgtcccctacggcaagcccctcaagtccaacggtaccggtatcgactcccaggtccagctcaagttcatggacctcgagcgatgggtctacctcctcgttctgctcatcggtgctgtcattgcccgatccgtcggtgttctggccttttgataaacatttcagtgcgattcgagtgagtagcacgataatagcccgtgtccatgtgctttaacacgtgtgctgtgctatttgaatatatcttatgaatgatacatgtaagtgacccgtagaccacagatcaacattccgagatgcatgcgaaatgtgaaaccaaggcaccggccggtacatctactccaaagtgtcgagatgtagtctgaagaatgtatatcaaccgtagaggtccatgttgacagaggttctacctgagaggctcattctgttgacttaagtgcacaactcctggcatcctcccctccaacacacaagttccgcaacgatcgaagcagagtcgagtacatccagatggcgctatcacgcgcatgaattaagctttaccgtcggggttacccggctga

SEQ ID NO: 26 - DNA, 20 bp, Artificial Sequence
  Nucleotide sequence of the genomic target of INT04
agaacacttcactccactat

SEQ ID NO: 27 - DNA, 6 bp, Artificial Sequence
  Nucleotide sequence of the inverted repeat of first 6 bp INT04 genomic target sequence
tgttct

SEQ ID NO: 28 - DNA, 50 bp, Artificial Sequence
  Nucleotide sequence of the 50 bp 5′ flank for targeting to INT04 integration locus
tcagccgacggactcagcggcctaatggacttcagaacacttcactccac

SEQ ID NO: 29 - DNA, 50 bp, Artificial Sequence
  Nucleotide sequence of connector D
aacgttgtccaggtttgtatccacgtgtgtccgttccgccaatattccgc

SEQ ID NO: 30 - DNA, 604 bp, Artificial Sequence
  Nucleotide sequence of promoter Yl_YP021
gtgctcttgcgctttctacgcagtttccattctctttctatgcacatctcggcaagtgtctcacatctttccttttcgcccacaaggactcacctgtacaagtatcagtacagcacgttgtgcaagcttcattagacagcaggcttaagtcatagacagtgtcgtattgctgttggattactggcagttaagcacttatttagactgacatttatactgtattctgctgagtctgagacagcgagctcatgtgtctttccatagcacagctacttatacagtagcaaatgtatcggaagtggcaaattggagcaagcgccagttattcgacattttttaatcagtggaggctttctataaaaaatgaaccatgcattttctcttgaaattcgttttgaaacccttgtcaatacaatagtacaagtacccatccagtccctctcaacaagtctagccgactcgtttccactgtgcctcctacacgcacatattgcgttatcttctaatgggttctctgtggaaccctaatccaccccgcaccccacaatgccatatatagaggagggcgcaatgtacttgttcctcaacacaacacgactacaaa

SEQ ID NO: 31 - DNA, 1134 bp, Artificial Sequence
  Nucleotide sequence of Xanthophyllomyces dendrorhous crtE gene
atggactacgccaacattctgaccgccatccctctcgagttcactccccaggacgacattgttctgctcgagccctaccactacctcggtaagaaccccggcaaggagatccgatcccagctcattgaggccttcaactactggctcgatgtcaagaaagaggatctcgaggtcatccagaacgttgttggtatgctccacaccgcctctctgctcatggacgatgtcgaggactcctccgttctgcgacgaggctcccccgttgcccacctcatctacggtatcccccagaccatcaacaccgccaactacgtctacttcctcgcctaccaggagatcttcaagctccgacccacccccatccccatgcccgtcattcctccctcctctgcttctctgcagtcctccgtctcctccgcttcttcctcctcttctgcttcttccgagaacggtggtacctccactcccaactcccagatccccttctccaaggacacctacctcgacaaggtcatcaccgacgagatgctgtctctgcaccgaggccagggtctggagctgttctggcgagactctctgacctgcccctccgaggaagagtacgtcaagatggttctcggcaagaccggtggcctcttccgaattgccgtccgactcatgatggccaagtccgagtgcgacattgactttgtccagctcgtcaacctcatctccatctacttccagatccgagacgactacatgaacctccagtcttccgagtacgcccacaacaagaactttgctgaggacctcaccgagggtaagttctccttccccaccatccactccatccacgccaacccctcttctcgactcgtcatcaacactctgcagaagaagtccacctcccccgagattctgcaccactgtgtcaactacatgcgaaccgagactcactccttcgagtacacccaggaagttctcaacaccctctccggtgctctcgagcgagagctcggccgactccagggtgagtttgccgaggccaactccaagattgacctcggtgatgtcgagtccgagggccgaaccggcaagaacgtcaagctcgaggccatcctcaagaagctcgctgacatccccctttaataa

SEQ ID NO: 32 - DNA, 304 bp, Artificial Sequence
  Nucleotide sequence of terminator Yl_ENO1
attacttttagttagctaatgtatgtatgattttgaccaggaactacgaatgcaaagcacaagttctacaagtagacgtgttgtatacctgataccatgttcttgtgtactcatgatcgtggacgtgttgttacatgttatatcaagcgtcatgattgcgactcataatggattgtgcgttattttgggtgaggtaattccaacaagtgttgggcaagcaattgtacgatagatcaagccatgactctacagtataatgaaaggagcgtgtgctcgttgtaggtactcaggtgaaataatcctc

SEQ ID NO: 33 - DNA, 50 bp, Artificial Sequence
  Nucleotide sequence of connector E
aaataaccacaaacatccttcccatatgctcggtcgtgcttgttgtacct

SEQ ID NO: 34 - DNA, 604 bp, Artificial Sequence
  Nucleotide sequence of promoter Yl_YP018
gtgcgaatcgaaagagccactgtttccctgctccctcaaaatatgcaattacaaatcatcgccatctacagacgcatatggacacatgcacatacttgcactccacagcaactcatgcgagtcttgcatgagtggcaaatggcagtggcgttagcaagagacgagatgcggcgtagataatcgatttgatgcacaccaattgtaacgttggcggtgaagcctatttggcaacccgccccgccacatggcattgtcgtaggctgggatttttagtccgaaattcgaggtggttttttgaagtgagacttgtttcttggaagtttaatgttttgacggggatgagtggaaagtggatggaaatttgaggtggtagaggtcggtcaattggtgagatttttgaataaatcaattctccagggaatttcgagctgattgccattttccgcatctttttacgacctcaaaacctcgagataattgcgtcagaaaaagaaaaacaatacctcgccacatgccaattaaccatcagataaccatatttaaaaagtatatataggtagtccagttcccacccctctgccaggtccccccaaccaccatcaaa

SEQ ID NO: 35 - DNA, 2025 bp, Artificial Sequence
  Nucleotide sequence of Xanthophyllomyces dendrorhous crtYB gene
atgaccgccctcgcctactaccagatccacctcatctacaccctccccatcctcggcctcctcggtctgctcacctcccccattctgaccaagttcgacatctacaagatctccatcctcgtcttcattgccttctccgccaccactccctgggactcctggatcatccgaaacggtgcctggacctacccctctgctgagtccggccagggtgtctttggtaccttcctcgatgtcccctacgaggagtacgctttctttgtcatccagaccgtcatcactggtctggtctacgttctcgccacccgacacctgctcccctctctcgccctccccaagacccgatcttccgctctgtctctggctctcaaggctctcatccccctgcccatcatctacctcttcaccgcccacccttccccctcccccgaccccctcgtcaccgaccactacttctacatgcgagctctgtccctcctcatcactccccctaccatgctcctcgctgctctgtccggtgagtacgcctttgactggaagtccggccgagccaagtccaccattgctgccatcatgatccccactgtctacctcatctgggtcgactacgttgccgtcggccaggactcttggtccatcaacgacgagaagattgtcggctggcgactcggcggtgttctccccatcgaagaggccatgttcttcctgctcaccaacctcatgattgttctcggcctctccgcctgcgaccacacccaggctctctacctcctccacggccgaaccatctacggcaacaagaagatgccctcttctttccccctcatcaccccacccgttctgtctctcttcttctcctctcgaccctactcctcccagcccaagcgagatctggagctcgccgtcaagctcctcgagaagaagtcccgatctttcttcgttgcctccgctggtttcccctccgaggtccgagagcgactcgtcggcctctacgccttctgccgagtcaccgatgatctcattgactcccccgaggtttcctccaacccccacgccaccattgacatggtttccgacttcctcaccctcctcttcggtcctcccctgcacccctctcagcccgacaagattctctcctctcccctgctgcctccctcccacccctcccgacccaccggtatgtaccccctgcctcccccgccctctctgtcccccgccgagctggtccagttcctcaccgagcgagtccccgtccagtaccactttgccttccgactgctggccaagctccagggtctgatcccccgataccccctcgacgagctcctgcgaggctacaccaccgacctcatcttccccctgtccaccgaggccgtccaggcccgaaagacccccattgagactactgctgacctcctcgactacggtctgtgtgttgccggctccgtcgccgagctcctcgtctacgtctcttgggcctctgctccctctcaggtccccgccactattgaggagcgagaggctgttctggttgcctcccgagagatgggtaccgccctccagctcgtcaacattgcccgagacatcaagggtgacgccaccgagggtcgattctacctccccctctccttcttcggtctgcgagacgagtccaagctcgccatccccaccgactggaccgagccccgacctcaggactttgacaagctgctctccctctccccctcctctaccctcccctcctccaacgcctccgagtccttccgatttgagtggaagacctactctctgcccctcgttgcttacgccgaggatctcgccaagcactcctacaagggtatcgaccgactccccactgaggttcaggccggtatgcgagctgcctgcgcctcttacctcctcattggccgagagatcaaggtcgtctggaagggtgatgtcggtgagcgacgaaccgttgccggttggcgacgagtccgaaaggtcctctccgtcgtcatgtccggttgggagggccagtaataa

SEQ ID NO: 36 - DNA, 304 bp, Artificial Sequence
  Nucleotide sequence of terminator Yl_POX2
acaagcgggtatttattgtatgaataaagattatgtattgattgcaaaaaagtgcatttgtagatgtggtttattgtagagagtacggtatgtactgtacgaacattaggagctacttctacaagtagattttcttaacaagggtgaaatttactaggaagtacatgcatatttcgttagtagaatcacaaaagaaatgtacaagcacgtactacttgtactccacaatgtggagtgggagcaaaaaaattggacgacaccggaatcgaaccggggacctcgcgcatgctaagcgcagcacctc

SEQ ID NO: 37 - DNA, 50 bp, Artificial Sequence
  Nucleotide sequence of connector F
gaaaccttcgaatccagccagcatgtcgacacccacaagatgtagtgcac

SEQ ID NO: 38 - DNA, 1004 bp, Artificial Sequence
  Nucleotide sequence of promoter Yl_ICL1
gtgcccttttgccagtatatccaccgcagcacccaccatgagcgacatctgatatcgtgccgcgaccactaccccaaataagctccaactaatatgccgaggcaggtgggaaactatgcactccagttgacgctgtagaagcacatggaaggtgcggaggcggtggcaacgaggggcatgagccatcaacgaggaaccacagacaaggcaaggggggaaacgcgaccggaatctctcgcggtcacgtgacccgcccgggttcactcgtccatgttgtgtctctggtgtcttcggccgactcgcattggttaaacttccaccaccgcaatcacgtcccactggccaaactttttctgctttctctgactttttctggccaaaaggcaacgtcggaaagggtcgggaggattcggaaccgacgaaaatcggccggctccagcgggggtagttcggcagtcctggtgggagcgctaggggagctgtggtctgtgtagggcgcgggtccgggtttgttgggtgtcaaatcacgtgtttttgcccccccgctgagccggactccgacaaccgtgtctccaacggcctgactaagctgctcccagcactctgccgtagcgttggtctgtcctgtcgcactctgttcaaagacagaagaaagaaaaagctaacctccacgtcagagacaatggtagaaggcttgttccttgcaaccgaggagagtgagtgttctcggcacgagcatcatgggcgatctggagggtatttttgaggggaaaaaacgggatcaggacaaacagaggccacagaccgggaatctgggccccaaaacggccttttcccgtcgcaaaaccggtctacatacaccccttcggcccgccacaggccggtgtgaaaaaccctaaagcttgcttcaaaccagacggacgcacagcaagacacatcatgaagagtcacctgcagtatatatagatctggggatccccagtagactgaccaagcatacaaaa

SEQ ID NO: 39 - DNA, 1752 bp, Artificial Sequence
  Nucleotide sequence of Xanthophyllomyces dendrorhous crtI gene
atgggcaaggagcaggaccaggacaagcctaccgccatcattgtcggctgtggtatcggtggtattgccactgctgctcgactcgccaaggaaggcttccaggtcaccgtctttgagaagaacgactactccggcggccgatgctctctcattgagcgagacggctaccgattcgaccagggcccctccctgctcctcctccccgatctcttcaagcagacctttgaggatctcggtgagaagatggaggactgggttgacctcatcaagtgtgagcccaactacgtctgccacttccacgacgaggagactttcaccttctccaccgacatggctctgctcaagcgagaggtcgagcgattcgagggcaaggacggctttgaccgattcctgtctttcatccaggaagcccaccgacactacgagctcgccgttgtccacgttctccagaagaacttccccggctttgccgccttcctgcgactccagttcatcggtcagatcctcgctctgcaccccttcgagtccatctggacccgagtctgccgatacttcaagaccgaccgactccgacgagtcttctccttcgccgttatgtacatgggccagtccccctactccgctcccggtacctactctctgctccagtacaccgagctcaccgagggtatctggtacccccgaggtggtttctggcaggtccccaacaccctcctccagatcgtcaagcgaaacaacccctccgccaagttcaacttcaacgcccccgtctcccaggttctcctctcccccgccaaggaccgagccaccggtgtccgacttgagtccggtgaggagcaccacgccgatgttgtcattgtcaacgccgacctcgtctacgcctccgagcacctcatccccgatgatgcccgaaacaagatcggtcagctcggtgaggtcaagcgatcttggtgggccgatctggtcggtggcaagaagctgaagggctcttgctcctctctgtccttctactggtctatggaccgaatcgtcgacggcctcggtggccacaacatcttcctggccgaggacttcaagggctccttcgacaccatcttcgaggagctcggcctccccgctgacccctccttctacgtcaacgtcccctctcgaatcgacccctctgctgctcctgagggtaaggacgccattgtcatcctcgtcccctgcggtcacattgatgcctccaacccccaggactacaacaagctcgttgcccgagcccgaaagtttgtcatccagaccctctccgctaagctcggtctgcccgacttcgagaagatgattgttgccgagaaggtccacgacgccccctcttgggagaaggagttcaacctcaaggacggttccatcctcggtctggcccacaacttcatgcaggttctcggcttccgaccctccacccgacaccccaagtacgacaagctgttctttgtcggtgcctccacccaccccggtactggtgtccccattgttctggctggtgccaagctcactgccaaccaggttctggagtccttcgaccgatctcctgctcccgaccccaacatgtctctctccgtcccctacggcaagcccctcaagtccaacggtaccggtatcgactcccaggtccagctcaagttcatggacctcgagcgatgggtctacctcctcgttctgctcatcggtgctgtcattgcccgatccgtcggtgttctggccttttgataa

SEQ ID NO: 40 - DNA, 304 bp, Artificial Sequence
  Nucleotide sequence of terminator Yl_TPI
acatttcagtgcgattcgagtgagtagcacgataatagcccgtgtccatgtgctttaacacgtgtgctgtgctatttgaatatatcttatgaatgatacatgtaagtgacccgtagaccacagatcaacattccgagatgcatgcgaaatgtgaaaccaaggcaccggccggtacatctactccaaagtgtcgagatgtagtctgaagaatgtatatcaaccgtagaggtccatgttgacagaggttctacctgagaggctcattctgttgacttaagtgcacaactcctggcatcctcccctc

SEQ ID NO: 41 - DNA, 50 bp, Artificial Sequence
  Nucleotide sequence of connector G
caacacacaagttccgcaacgatcgaagcagagtcgagtacatccagatg

SEQ ID NO: 42 - DNA, 50 bp, Artificial Sequence
  Nucleotide sequence of the 50 bp 3′ flank for targeting to INT04 integration locus
gcgctatcacgcgcatgaattaagctttaccgtcggggttacccggctga

SEQ ID NO: 43 - DNA, 24 bp, Artificial Sequence
  Nucleotide sequence of the forward primer to confirm targeted integration at INT05 locus
cattgatcttactgtcgaatgtac

SEQ ID NO: 44 - DNA, 21 bp, Artificial Sequence
  Nucleotide sequence of the reverse primer to confirm targeted integration at INT05 locus
ttggcgtaggtgccgatgtag

SEQ ID NO: 45 - DNA, 21 bp, Artificial Sequence
  Nucleotide sequence of the forward primer to confirm targeted integration at INT04 locus
ctgcggtgtcattggtcttac

SEQ ID NO: 46 - DNA, 18 bp, Artificial Sequence
  Nucleotide sequence of the reverse primer to confirm targeted integration at INT04 locus
cgtattggcgagaacaag

SEQ ID NO: 47 - DNA, 20 bp, Artificial Sequence
  Nucleotide sequence of the reverse primer annealing to connector D (COND)
gcggaatattggcggaacgg

SEQ ID NO: 48 - DNA, 20 bp, Artificial Sequence
  Nucleotide sequence of the reverse primer annealing to connector E (CONE)
aggtacaacaagcacgaccg

SEQ ID NO: 49 - DNA, 20 bp, Artificial Sequence
  Nucleotide sequence of the forward primer annealing to Yl_ENO1.ter
cgtgtgctcgttgtaggtac

SEQ ID NO: 50 - DNA, 22 bp, Artificial Sequence
  Nucleotide sequence of the reverse primer annealing to Yl_YP018.pro
tatgtgcatgtgtccatatgcg

SEQ ID NO: 51 - DNA, 21 bp, Artificial Sequence
  Nucleotide sequence of the forward primer annealing to Yl_POX2.ter
cacgtactacttgtactccac

SEQ ID NO: 52 - DNA, 21 bp, Artificial Sequence
  Nucleotide sequence of the reverse primer annealing to Yl_ICL1.pro
cttgtctgtggttcctcgttg

SEQ ID NO: 53 - DNA, 22 bp, Artificial Sequence
  Nucleotide sequence of the forward primer annealing to connector F (CONF)
gaaaccttcgaatccagccagc

SEQ ID NO: 54 - DNA, 21 bp, Artificial Sequence
  Nucleotide sequence of the forward primer annealing to connector G (CONG)
caacacacaagttccgcaacg

SEQ ID NO: 55 - DNA, 12810 bp, Artificial Sequence
  Nucleotide sequence of plasmid pSTV085
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgtgttctctgatgagtccgtgaggacgaaacgagtaagctcgtcagaacacttcactccactatgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 56 - DNA, 12810 bp, Artificial Sequence
  Nucleotide sequence of plasmid pSTV089
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgcgatatctgatgagtccgtgaggacgaaacgagtaagctcgtcatatcgccgcaagattacacgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 57 - DNA, 6 bp, Artificial Sequence
  Nucleotide sequence of 6 bp inverted repeat of the KU70 genomic target
cgatat

SEQ ID NO: 58 - DNA, 20 bp, Artificial Sequence
  Nucleotide sequence of the KU70 genomic target
atatcgccgcaagattacac

SEQ ID NO: 59 - DNA, 100 bp, Artificial Sequence
  Nucleotide sequence of the 100-bp donor DNA fragment used for knocking out the KU70 gene in the Yarrowia genome
catcgcgacacgaacacgaaacacgaaccacgaaccgccgctttttgaaactagggaggcacatctaaacgaataacgaatattaatgataccatcatat

SEQ ID NO: 60 - DNA, 20 bp, Artificial Sequence
  Nucleotide sequence of the forward primer to confirm knock out of KU70 gene in the Yarrowia genome
gagcacgtcacgtgttctcc

SEQ ID NO: 61 - DNA, 20 bp, Artificial Sequence
  Nucleotide sequence of the reverse primer to confirm knock out of KU70 gene in the Yarrowia genome
agtggtaccgctactttgtg

(19) United States
(12) Patent Application Publication
(10) Pub. No.: US 2022/0235378 A1
(43) Pub. Date: Jul. 28, 2022

(54) MULTIPARTITE CRISPR DONOR

(71) Applicant: DSM IP Assets B.V., Heerlen (NL)

(72) Inventors: Brenda VONK, Echt (NL); Adriana Christina VAN DER HOEK, Echt (NL); Priscilla ZWARTJENS, Echt (NL); Johannes Andries ROUBOS, Echt (NL)

(21) Appl. No.: 17/608,429

(22) PCT Filed: Apr. 23, 2020
(86) PCT No.: PCT/EP2020/061406

§ 371 Date: Nov. 2, 2021

(30) Foreign Application Priority Data
May 6, 2019 (EP) 19172841.9

(51) Int. Cl. C12N 15/90

(52) U.S. Cl. CPC C12N 15/905

(57) ABSTRACT

The present invention relates to the field of molecular biology and cell biology. More specifically, the present invention relates to a CRISPR-assembly gene editing system in a eukaryotic cell.

### FIELD OF THE INVENTION

The present invention relates to the field of molecular biology and cell biology. More specifically, the present invention relates to a CRISPR-assembly gene editing system in a eukaryotic cell.

### BACKGROUND TO THE INVENTION

A polynucleotide-guided nuclease system, also referred to as polynucleotide-guided genome editing system, from which the best known are the CRISPR/Cas9 and CRISPR/Cpf1 systems, is a powerful tool that has been leveraged for genome editing and gene regulation, e.g. to generate within a host cell a targeted mutation, a targeted insertion or a targeted deletion/knock-out. This tool requires at least a polynucleotide-guided nuclease such as Cas9 and Cpf1 and a guide-polynucleotide such as a guide-RNA that enables the genome editing enzyme to target a specific sequence of DNA. In addition, for editing of the genome in a precise way, a donor polynucleotide such as a donor DNA is mostly required, especially when relying on homologous recombination for editing precisely at a desired spot in the genome instead of relying on repair by a random repair process, such as non-homologous end joining. For each target site, a donor polynucleotide needs to be designed and synthesized. For targeted modification with a polynucleotide-guided genome editing system, a donor polynucleotide which is specific for a target need to be used. Especially for multiplex approaches such as when screening, e.g., a knock-out library, a knock-down library or a promoter-replacement library, the experimental work is quite laborious since each specific donor will have be synthesized and transformed. For screening multiple targets and/or multiple modifications in one experiment, the state of the art set-up requires a multitude of polynucleotides to be added and used and an even higher amount of screenings for a cell comprising the desired properties. Accordingly, there is a continuing urge to develop improved and simplified and donor polynucleotide tools.

The invention addresses above described need and provides such technique.

### SUMMARY OF THE INVENTION

Provided is a method for genome editing within a cell comprising,

contacting the cell with at least two double-stranded polynucleotides such that the at least two double-stranded polynucleotides are introduced into the cell,

wherein a part of the first of the at least two double-stranded polynucleotides has sequence identity with a part of the second of the at least two double-stranded polynucleotides, such that, within the cell, the at least two double-stranded polynucleotides can assemble into a double-stranded polynucleotide construct,

wherein the double-stranded polynucleotide construct has at its 5′-end sequence identity with the genome of the cell within the proximity of a break in the genome of the cell and wherein the double-stranded polynucleotide construct has at its 3-end sequence identity with the genome of the cell within the proximity of a break in the genome of the cell, and

wherein the double-stranded polynucleotide construct integrates into the genome of the cell within the proximity of the break in the genome of the cell.

Further provided is a composition comprising the cell defined here above and at least one of at least two double-stranded polynucleotides according to the invention.

Further provided is a cell comprising an assembled double-stranded polynucleotide construct obtainable by a method according to the invention.

Further provided is a cell obtainable by or produced by a method according to the invention, further comprising a polynucleotide encoding a compound of interest.

Further provided is a method for the production of a compound of interest, comprising culturing the cell defined here above under conditions conducive to the production of the compound of interest, and, optionally, purifying or isolating the compound of interest.

Further provided is the use of a plurality of double-stranded polynucleotides in genome editing, wherein parts of the members of the plurality of double-stranded polynucleotides have sequence identity with parts of other members such that they can, within a cell, assemble into a double-stranded polynucleotide construct and wherein the double-stranded polynucleotide construct(s) can integrate into the genome of the cell within the proximity of a break into the genome.

### BRIEF DESCRIPTION OF THE DRAWINGS

FIG. 1 depicts the vector map of the single copy (CEN/ARS) vector MB7452 encoding Cas9 codon optimized for expression in Yarrowia lipolytica. Codon optimized Cas9 is expressed from the Yarrowia lipolytica 007 promoter and the Yarrowia lipolytica GPD terminator. A NatMX marker cassette is present on the vector, which confers resistance against nourseothricin to allow selection of transformants on agar plate or in liquid cultures. The beta lactamase marker allows for selection of the plasmid in E. coli.

FIG. 2 depicts the vector map of vector pSTV086. A HygB marker cassette is present on the vector, which confers resistance against hygromycin B to allow selection of transformants on agar plate or in liquid cultures. The vector expresses Cas9 (codon optimized for expression in Yarrowia lipolytica) as well as the sgRNA expression cassette targeting INT05. The sgRNA expression cassette comprises Yarrowia YI_HYPO promoter, 6 bp inverted repeat of the INT05 genomic target, HH ribozyme, INT05 genomic target, HDV ribozyme and PGM terminator.

FIG. 3 depicts the vector map of vector pSTV085. A HygB marker cassette is present on the vector, which confers resistance against hygromycin B to allow selection of transformants on agar plate or in liquid cultures. The vector expresses Cas9 (codon optimized for expression in Yarrowia lipolytica) as well as the sgRNA expression cassette targeting INT04. The sgRNA expression cassette comprises Yarrowia YI_HYPO promoter, 6 bp inverted repeat of the INT04 genomic target, HH ribozyme, INT04 genomic target, HDV ribozyme and PGM terminator.

FIG. 4 depicts in vivo assembly and integration of the ctrE, crtYB and crtl expression cassettes in the INT04 locus of the Yarrowia genome. 5′ and 3′ represent the genomic FLANK sequences for targeted integration at the INT04 locus. D, E, F and G represent 50-bp connector sequences, which are non-coding DNA sequences added to the expression cassettes. The CRISPR-Cas9 cut at the INT04 locus is represented by the black triangle.

FIG. 5 depicts the PCR approach to confirm correct integration and assembly of the ctrE, crtYB and crtl expression cassettes at the INT04 locus. D, E, F and G represent 50-bp connector sequences, which are non-coding DNA sequences added to the expression cassettes.

FIG. 6 depicts the vector map of vector pSTV089. A HygB marker cassette is present on the vector, which confers resistance against hygromycin B to allow selection of transformants on agar plate or in liquid cultures. The vector expresses Cas9 (codon optimized for expression in Yarrowia lipolytica) as well as the sgRNA expression cassette targeting the KU70 gene. The sgRNA expression cassette comprises Yarrowia YI_HYPO promoter, 6 bp inverted repeat of the KU70 genomic target, HH ribozyme, KU70 genomic target, HDV ribozyme and the Yarrowia YI_PGM terminator.

### DESCRIPTION OF THE SEQUENCES

SEQ ID NO: 1 sets out the nucleotide sequence of Cas9, including a C-terminal SV40 nuclear localization signal, codon pair optimized for expression in Yarrowia lipolytica. The sequence includes the 007 promoter sequence and the GPD terminator sequence, both from Yarrowia lipolytica.

SEQ ID NO: 2 sets out the nucleotide sequence of vector MB7452

SEQ ID NO: 3 sets out the nucleotide sequence of vector pSTV086 (all in one).

SEQ ID NO: 4 sets out the nucleotide sequence of the cassette of the donor DNA for expression of GFP (YI_HSP.pro-A.vic_eGFP ORF-YI_GPD.ter). The donor DNA fragment has 50 bp genomic DNA flanks on either side for targeted integration in the INT05 locus.

SEQ ID NO: 5 sets out the nucleotide sequence of the 5′ bipartite donor DNA fragment, comprising the 50 bp genomic sequence of INT05, the YI_HSP.promoter and part of the A.vic_eGFP ORF

SEQ ID NO: 6 sets out the nucleotide sequence of the 3′ bipartite donor DNA fragment, comprising part of the YI_HSP promoter—A.vic_eGFP ORF-YI.GPD terminator and 50 bp genomic sequence of INT05

SEQ ID NO: 7 sets out the nucleotide sequence of the genomic target of integration locus INT05 in the Yarrowia lipolytica genome

SEQ ID NO: 8 sets out the nucleotide sequence of the integration locus INT05.

SEQ ID NO: 9 sets out the nucleotide sequence of 6 bp inverted repeat of the INT05 genomic target.

SEQ ID NO: 10 sets out the nucleotide sequence of HH ribozyme

SEQ ID NO: 11 sets out the nucleotide sequence of HDV ribozyme

SEQ ID NO: 12 sets out the nucleotide sequence of YI_HYPO promoter

SEQ ID NO: 13 sets out the nucleotide sequence of YI_GPD terminator

SEQ ID NO: 14 sets out the nucleotide sequence of the YI_PGM terminator

SEQ ID NO: 15 sets out the nucleotide sequence of the YI_007 promoter

SEQ ID NO: 16 sets out the nucleotide sequence of the YI_HSP promoter

SEQ ID NO: 17 sets out the nucleotide sequence of the forward primer for amplification of donor DNA fragment: INT05 5′ FLANK-YI_HSP.pro-A.vic_eGFP ORF-YI_GPD.ter—INT05 3′ FLANK.

SEQ ID NO: 18 sets out the nucleotide sequence of the reverse primer for amplification of donor DNA fragment: INT05 5′ FLANK-YI_HSP.pro-A.vic_eGFP ORF-YI_GPD.ter—INT05 3′ FLANK.

SEQ ID NO: 19 sets out the nucleotide sequence of the forward primer for amplification of the 5′ bipartite donor DNA fragment

SEQ ID NO: 20 sets out the nucleotide sequence of the reverse primer for amplification of the 5′ bipartite donor DNA fragment

SEQ ID NO: 21 sets out the nucleotide sequence of the forward primer for amplification of the 3′ bipartite donor DNA fragment

SEQ ID NO: 22 sets out the nucleotide sequence of the reverse primer for amplification of the 3′ bipartite donor DNA fragment

SEQ ID NO: 23 sets out the nucleotide sequence of the donor DNA fragment which comprises the crtE expression cassette. The crtE expression cassette is flanked by a 50 bp INT04 genomic sequence on the 5′ side for targeted integration at INT04 locus and on the 3′ side a 50 bp connector sequence, CONE, is present for in vivo assembly with the crtYB expression cassette.

SEQ ID NO: 24 sets out the nucleotide sequence of the donor DNA fragment which comprises the crtYB expression cassette. The crtYB expression cassette is flanked by a 50 bp connector sequence; CONE, on the 5′ side and CONF on the 3′ side. The connector sequences are for in vivo assembly of the donor DNA cassettes used in transformation. The crtYB will be assembled with the crtE expression cassette donor DNA (SEQ ID NO:23) on the 5′ side and the crt/expression cassette donor DNA (SEQ ID NO:25) on the 3′ side.

SEQ ID NO: 25 sets out the nucleotide sequence of the donor DNA fragment which comprises the crtl expression cassette. The crtl expression cassette is flanked by a 50 bp connector sequence, CONF, on the 5′ side for in vivo assembly with the crtYB expression cassette and on the 3′ side a 50 bp INT04 genomic sequence for targeted integration at INT04.

SEQ ID NO: 26 sets out the nucleotide sequence of the genomic target of INT04

SEQ ID NO: 27 sets out the nucleotide sequence of the inverted repeat of first 6 bp INT04 genomic target-sequence.

SEQ ID NO: 28 sets out the nucleotide sequence of the 50 bp 5′ flank for targeting to INT04 integration locus.

SEQ ID NO: 29 sets out the nucleotide sequence of connector D

SEQ ID NO: 30 sets out the nucleotide sequence of promoter YI_YPO21

SEQ ID NO: 31 sets out the nucleotide sequence of Xanthophyllomyces dendrorhous crtE gene

SEQ ID NO: 32 sets out the nucleotide sequence of terminator YI_ENO1

SEQ ID NO: 33 sets out the nucleotide sequence of connector E

SEQ ID NO: 34 sets out the nucleotide sequence of promoter YI_YP018

SEQ ID NO: 35 sets out the nucleotide sequence of Xanthophyllomyces dendrorhous crtYB gene

SEQ ID NO: 36 sets out the nucleotide sequence of terminator YI_POX2

SEQ ID NO: 37 sets out the nucleotide sequence of connector F

SEQ ID NO: 38 sets out the nucleotide sequence of promoter YI_ICL1

SEQ ID NO: 39 sets out the nucleotide sequence of Xanthophyllomyces dendrorhous crtl gene

SEQ ID NO: 40 sets out the nucleotide sequence of terminator YI_TPI

SEQ ID NO: 41 sets out the nucleotide sequence of connector G

SEQ ID NO: 42 sets out the nucleotide sequence of the 50 bp 3′ flank for targeting to INT04 integration locus.

SEQ ID NO: 43 sets out the nucleotide sequence of the forward primer to confirm targeted integration at INT05 locus.

SEQ ID NO: 44 sets out the nucleotide sequence of the reverse primer to confirm targeted integration at INT05 locus.

SEQ ID NO: 45 sets out the nucleotide sequence of the forward primer to confirm targeted integration at INT04 locus.

SEQ ID NO: 46 sets out the nucleotide sequence of the reverse primer to confirm targeted integration at INT04 locus.

SEQ ID NO: 47 sets out the nucleotide sequence of the reverse primer annealing to connector D (COND)

SEQ ID NO: 48 sets out the nucleotide sequence of the reverse primer annealing to connector E (CONE)

SEQ ID NO: 49 sets out the nucleotide sequence of the forward primer annealing to YI_ENO1.ter

SEQ ID NO: 50 sets out the nucleotide sequence of the reverse primer annealing to YI_YP018.pro

SEQ ID NO: 51 sets out the nucleotide sequence of the forward primer annealing to YI_POX2.ter

SEQ ID NO: 52 sets out the nucleotide sequence of the reverse primer annealing to YI_ICL1.pro

SEQ ID NO: 53 sets out the nucleotide sequence of the forward primer annealing to connector F (CONF)

SEQ ID NO: 54 sets out the nucleotide sequence of the forward primer annealing to connector G (CONG)

SEQ ID NO: 55 sets out the nucleotide sequence of plasmid pSTV085

SEQ ID NO: 56 sets out the nucleotide sequence of plasmid pSTV089

SEQ ID NO: 57 sets out the nucleotide sequence of 6 bp inverted repeat of the KU70 genomic target

SEQ ID NO: 58 sets out the nucleotide sequence of the KU70 genomic target

SEQ ID NO: 59 sets out the nucleotide sequence of the 100-bp donor DNA fragment used for knocking out the KU70 gene in the Yarrowia genome

SEQ ID NO: 60 sets out the nucleotide sequence of the forward primer to confirm knock out of KU70 gene in the Yarrowia genome

SEQ ID NO: 61 sets out the nucleotide sequence of the reverse primer to confirm knock out of KU70 gene in the Yarrowia genome

### DETAILED DESCRIPTION OF THE INVENTION

In a first aspect, there is provided for a method for genome editing within a cell comprising, contacting the cell with at least two double-stranded polynucleotides such that the at least two double-stranded polynucleotides are introduced into the cell,

wherein a part of the first of the at least two double-stranded polynucleotides has sequence identity with a part of the second of the at least two double-stranded polynucleotides, such that, within the cell, the at least two double-stranded polynucleotides can assemble into a double-stranded polynucleotide construct,

wherein the double-stranded polynucleotide construct has at its 5′-end sequence identity with the genome of the cell within the proximity of a break in the genome of the cell and wherein the double-stranded polynucleotide construct has at its 3′-end sequence identity with the genome of the cell within the proximity of a break in the genome of the cell, and

wherein the double-stranded polynucleotide construct integrates into the genome of the cell within the proximity of the break in the genome of the cell.

The method for genome editing is herein referred to as a method according to the invention. A polynucleotide and a cell are defined in the section “Definitions” herein. The terms “assembly” and interchangeably “assembly within a cell” mean that the two or more double-stranded polynucleotides aggregate together within a cell by base paring to form a single construct, which construct is processed by the cell into a double-stranded polynucleotide construct. The at least two double stranded polynucleotides may be contacted with cell in any which way known the person skilled in the art in order to introduce the least two double stranded polynucleotides into the cell. The double-stranded polynucleotide construct is herein also referred to as the double-stranded polynucleotide construct according to the invention and is interchangeably referred to as “donor”, “donor polynucleotide” or “donor DNA”, if the double-stranded polynucleotide construct is a DNA. The proximity of a break in the genome of a cell is herein defined as within at least 5 nucleotides from the break, such as 5, 4, 3, 2, or 1 nucleotides from the break, or such as within at least 10, 20, 30, 40, 50, 60, 70, 80, 90, 100, nucleotides from the break. In an embodiment, within the proximity of the break in the genome is at the break in the genome of the cell. Integration into the genome of the cell within the proximity of the break in the genome of the cell is consequently within at least 5 nucleotides from the break, such as 5, 4, 3, 2, or 1 nucleotides from the break, or such as within at least 10, 20, 30, 40, 50, 60, 70, 80, 90, 100, nucleotides from the break.

The at least two double-stranded polynucleotides have sequence identity with each other (a part of the first of the at least two double-stranded polynucleotides has sequence identity with a part of the second of the at least two double-stranded polynucleotides), such that, within the cell, the at least two double-stranded polynucleotides can assemble into a double-stranded polynucleotide construct. The parts having sequence identity preferably have at length of at least 5, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. The parts having sequence identity preferably have a length of 5, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. The degree of complementarity, when optimally aligned using a suitable alignment algorithm, is preferably higher than 50%, 60%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity. The sequence identity may be 100%. Parts having sequence identity may herein be referred to as overlapping parts.

The double-stranded polynucleotide construct or donor has sequence identity with the genome of the cell within the proximity of a break in the genome at both its 5′-end and at its 3′-end. This allows the donor to integrate into the genome within the proximity of the break in the genome by homologous recombination. The 5′-end and 3′-end of the donor having sequence identity with the genome of the cell within the proximity of a break in the genome may herein also be referred to as flanks, e.g. 5′-flank and 3′-flank. The 5′-flank and 3′-flank according to the invention may have any length as long as allowing recombination in vivo such as within a cell such that the self-guiding integration construct as disclosed herein integrates into the target genome. A 5′-flank may have a length of at least 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900 or 1000 nucleotides. A 5′-flank may have a length of at most 1000, 900, 800, 700, 600, 500, 450, 400, 350, 300, 250, 200, 190, 180, 170, 160, 150, 140, 130, 120, 110, 100, 95, 90, 85, 80, 75, 70, 65, 60, 55, 50, 45, 40, 35, 30 or 25 nucleotides.

A 3′-flank may have a length of at least 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900 or 1000 nucleotides.

A 3′-flank may have a length of at most 1000, 900, 800, 700, 600, 500, 450, 400, 350, 300, 250, 200, 190, 180, 170, 160, 150, 140, 130, 120, 110, 100, 95, 90, 85, 80, 75, 70, 65, 60, 55, 50, 45, 40, 35, 30 or 25 nucleotides. A 5′-flank may have a length of from about 25 to about 80 nucleotides, more preferably from about 30 to about 80 nucleotides, more preferably from about 50 to about 80 nucleotides. A 3′-flank may have a length of from about 25 to about 80 nucleotides, more preferably from about 30 to about 80 nucleotides, more preferably from about 50 to about 80 nucleotides.

A 5′-flank may have a length of from 25 to 80 nucleotides, more preferably from 30 to 80 nucleotides, more preferably from 50 to 80 nucleotides. A 3′-flank may have a length of from 25 to 80 nucleotides, more preferably from 30 to 80 nucleotides, more preferably from 50 to 80 nucleotides. A 5′-flank may have a length of from 25 to 80 nucleotides, such as 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80 nucleotides. A 3′-flank may have a length of from 15 to 80 nucleotides, such as 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80 nucleotides.

The double-stranded polynucleotide construct (donor polynucleotide) may be any suitable additional polynucleotide, functional or non-functional, such as a control sequence, a marker, a gene of interest encoding a compound of interest as defined elsewhere herein, or a disruption construct. The control sequence may be any control sequence or combination of control sequences, such as a promotor, a KOZAK sequence, a signal sequence, a terminator, a pre-sequence, a pre-pro-sequence, a leader sequence, an activator sequence, a repressor sequence, a HIS-tag, a split-GFP tag or any other N-terminal tag. A preferred control sequence is a promoter sequence. This e.g. enables to insert a promoter or to replace an endogenous promoter, or a part thereof, by another promoter. The introduced promoter may be stronger or weaker than the endogenous promoter and/or may be an inducible promoter. Such promoters are known to the person skilled in the art. The marker may be any type of marker as long as it can be identified and thus serves as a marker. The marker may e.g. be a selection marker or may e.g. be an identifiable polynucleotide with known sequence to be used as a barcode or may be a tag such as a HIS-tag, GFP-tag, split GFP-tag, solubility tag. The gene of interest may be any gene of interest and is preferably one as defined in the section “General Definitions”. The gene of interest may be a complete expression construct comprising a promoter, a coding sequence and a terminator, or may at least comprise a coding sequence. The donor polynucleotide may be a polynucleotide to generate within the cell a targeted mutation, a targeted insertion or a targeted deletion/knock-out. The terms “targeted mutation”, “targeted insertion” and “targeted deletion/knock-out” in all embodiments of the invention mean that the mutation, insertion, deletion/knock-out is made in a pre-defined place in the genome of the host cell. A mutation can be a silent mutation or a mutation that results in an amino acid change. A mutation is not limited to mutation of a single nucleotide, two or more nucleotides may be mutated. An insertion means that at least one nucleotide is added to the target genome. An insertion can be combined with a mutation and/or a deletion as long the resulting genome is different from the target genome before editing. A deletion means that at least one nucleotide is deleted from the target genome. A deletion can be combined with a mutation and/or deletion as long as the resulting genome is different from the target genome before editing.

An insertion may have any suitable length, such as at least one nucleotide, at least 10 nucleotides, at least 20, 30, 40, 50, 60, 70, 80, 90, 100, 110, 120, 130, 140, 150, 160, 170, 180, 190, 200, 250, 300, 400, 500, 600, 700, 800, 900, or at least 1000 nucleotides. An insertion may have at most 20, 30, 40, 50, 60, 70, 80, 90, 100, 110, 120, 130, 140, 150, 160, 170, 180, 190, 200, 250, 300, 400, 500, 600, 700, 800, 900, or at least 1000 nucleotides. An insertion may be within the range of 20-1000, 100-1000, 100-500, or 200-500 nucleotides. A deletion may have any suitable length, such as at least one, two, three, four, five, six, seven, eight, nine nucleotide(s), at least 10 nucleotides, at least 20, 30, 40, 50, 60, 70, 80, 90, 100, 110, 120, 130, 140, 150, 160, 170, 180, 190, 200, 250, 300, 400, 500, 600, 700, 800, 900, or at least 1000 nucleotides. A deletion may be at most 20, 30, 40, 50, 60, 70, 80, 90, 100, 110, 120, 130, 140, 150, 160, 170, 180,190, 200, 250, 300, 400, 500, 600, 700, 800, 900, 1000, 2000, 3000, 4000 or 5000 nucleotides. A deletion may be within the range of 20-5000, 100-1000, 100-500, or 200-500 nucleotides.

Conveniently, more than two double-stranded polynucleotides can be used in the method according to the invention. Accordingly, there is provided for a method according to the invention, wherein at least three double-stranded polynucleotides are introduced into the cell,

wherein a part of the first of the at least three double-stranded polynucleotides has sequence identity with a part of the second of the at least three double-stranded polynucleotides and wherein a part of the second of the at least three double-stranded polynucleotides has sequence identity with the third of the at least three double-stranded polynucleotides, such that, within the cell, the at least three double-stranded polynucleotides can assemble into a double-stranded polynucleotide construct,

wherein the double-stranded polynucleotide construct has at its 5′-end sequence identity with the genome of the cell within the proximity of the break in the genome of the cell and wherein the double-stranded polynucleotide construct has at its 3′-end sequence identity with the genome of the cell within the proximity of the break in the genome of the cell, and

wherein the double-stranded polynucleotide construct integrates into the genome of the cell within the proximity of the break in the genome of the cell.

Moreover, there is provided for a method according to the invention wherein, at least four, such as five, six, seven, eight, nine, ten, eleven, twelve, thirteen, fourteen, fifteen, sixteen, seventeen or eighteen double-stranded polynucleotides are introduced into the cell that are capable of assembly into the double-stranded polynucleotide construct.

As set forward here above, the double-stranded polynucleotide construct may comprise a coding sequence, e.g. encoding a compound of interest. It may comprise part of a coding sequence, a complete coding sequence, a gene, or multiple coding sequences, such as two, three, four, five, six, seven or eight coding sequences.

In an embodiment, the double-stranded polynucleotide construct does not encode a guide-polynucleotide or a part thereof, such as a guide-RNA molecule or a part thereof.

Conveniently, a plurality of double-stranded polynucleotides can be introduced into the cell and assembles in a library of distinct double-stranded polynucleotide constructs, wherein each double-stranded polynucleotide construct is assembled from at least two double-stranded polynucleotides.

It enables e.g. a multiplex or library of completely distinct donor polynucleotides. It also enables e.g. a library of donor polynucleotides wherein parts of the donor are identical, such as control sequences and wherein the coding sequences are different. It also enables e.g. a library of donor polynucleotides wherein parts of the donor are identical, such as the coding sequence and wherein the control sequences are different. In such multiplex or library, at least two, such as three, four, five, six, seven or more distinct double-stranded polynucleotide constructs may be comprised. The distinct double-stranded polynucleotide constructs may each integrate into a distinct locus in the genome of the cell.

In an embodiment, at least one of the double-stranded polynucleotide constructs comprises a marker. Such marker may be any selectable or detectable marker. A selectable marker may a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof. A detectable marker may be a bar code or the like. Conveniently, the method according to the invention may be performed iteratively, such as twice, three of four times, wherein in its iteration one or more distinct double-stranded polynucleotide constructs are assembled and integrated into the genome of the cell. Such iterative method may be facilitated by the use of a different selectable marker in each iteration, or the cell may be cured of the marker after each iteration.

In a specific embodiment, the assembly of the double-stranded polynucleotide construct may facilitated by a least one connector oligonucleotide that has sequence identity with part of two double-stranded polynucleotides. In such case, e.g. the at least two double-stranded polynucleotides do not need to overlap, a connector oligonucleotide has sequence identity with the 3-end of one of the at least two double-stranded polynucleotides and with the 5′-end of another of the at least two double-stranded polynucleotides, such that the at least two double-stranded polynucleotides can assembly into a double-stranded polynucleotide construct.

In another specific embodiment of the invention, the 5′-flank and/or the 3-flank having sequence identity with the target genome in the proximity of the break in the genome, are located on separate single-stranded or double-stranded oligonucleotides (also referred to as ssODN's and dsODN's, respectively; see EP16181781.2, which is herein incorporated by reference). In such case, a single-stranded or double-stranded oligonucleotide has a part (i.e. a portion of polynucleotide sequence) that has sequence identity with the part of the double stranded polynucleotide construct and has a part that has sequence identity with a sequence within the proximity of a break in the genome of the cell. In a typical example, to which the invention is not limited, a first single-stranded or double stranded oligonucleotide has a part that has sequence identity with a sequence on the 5′-end of the part of the double stranded polynucleotide construct and has a part that has sequence identity with a sequence in the genome that is located 5′ of the break in the genome of the cell; and, a second single-stranded or double stranded oligonucleotide has a part that has sequence identity with a sequence on the 3′-end of the part of the double-stranded polynucleotide construct and has a part that has sequence identity with a sequence in the genome that is located 3′ of the break in the genome of the cell. In this specific embodiment applying to all embodiments of the invention, the single-stranded oligonucleotide(s) and/or double-stranded oligonucleotide(s) mediate the in vivo (within a cell) integration of the self-guiding integration construct into the target genome. In this specific embodiment that may be applied to all embodiments of the invention, the teachings of WO2017037304 on in vitro assembly of a polynucleotide construct can conveniently be used.

The cell in the method according to the invention, may be any cell, e.g. such as defined in the section “General Definitions” herein. Such cell may be a eukaryotic cell, such as a fungal cell, such as a non-conventional yeast cell, such as a Yarrowia cell, such as a Yarrowia lipolytica cell.

In the method according to the invention, the cell may be deficient in an NHEJ (non-homologous end joining) component such as defined in the section “General Definitions” herein.

In the method according to the invention, the break in the genome may be one selected from the group consisting of a single-stranded break (nick), an induced single-stranded break, a double-stranded break and an induced double-stranded break.

In the method according to the invention, the break may be induced by a functional genome editing system. The functional genome editing system may be any suitable system known to the person skilled in the art. Suitable functional genome editing systems for use in all embodiments of the invention include: RNA-guided endonucleases like CRISPR/Cas (Mali et al., 2013; Cong et al., 2013) or CRISPR/Cpf1 (Zetsche et al., 2015). The functional genome editing enzyme can be a native or a heterologous enzyme, and can be an enzyme such as a Cas enzyme, preferably Cas9 or Cas9 nickase; a Cpf1.

In the method according to the invention, the cell may express a functional heterologous genome editing enzyme, such as a Cas enzyme, such as Cas9 or Cas9 nickase; Cpf1; I-Scel, or in the cell a heterologous genome editing enzyme, such as a Cas enzyme, such as Cas9 or Cas9 nickase; Cpf1; I-Scel, may be present.

In the method according to the invention, a guide-polynucleotide such as a guide-RNA may be present within the cell. A guide-polynucleotide is preferably a functional guide-polynucleotide. Guide polynucleotides have been described extensively and are known to the person skilled in the art (e.g. Mali et al., 2013; Cong et al., 2013; Zetsche et al., 2015; Gao and Zhao, 2014). A preferred functional guide-polynucleotide is a functional guide-RNA. A functional guide-RNA comprises at least a guide-sequence. A guide-sequence herein is a part of the guide-RNA that is able to hybridize with a target-sequence in a target-polynucleotide such as a target-genome and is able to direct sequence-specific binding of a genome editing system to the target-polynucleotide. The guide-RNA is a polynucleotide according to the general definition of a polynucleotide set out herein. A guide-sequence is herein also referred as a target-sequence and is essentially the complement of a target-polynucleotide such that the guide-polynucleotide is able to hybridize with the target-polynucleotide, preferably under physiological conditions in a host cell. The degree of complementarity, when optimally aligned using a suitable alignment algorithm, is preferably higher than 50%, 60%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity. The sequence identity may be 100%.

In the method according to the invention, the guide-polynucleotide in the cell is expressed from a vector, preferably a plasmid, preferably the vector is introduced into the cell together with at least one of the at least two double-stranded polynucleotides. Methods to express guide-polynucleotides are known to the person skilled in the art (see e.g. Mali et al., 2013; Cong et al., 2013; Zetsche et al., 2015; Gao and Zhao, 2014).

In the method according to the invention, the plasmid from which the guide-polynucleotide is expressed, is assembled within the cell by integration of a single-stranded or double-stranded oligonucleotide comprising the target-sequence of the guide-polynucleotide into the plasmid, wherein the single-stranded or double-stranded oligonucleotide comprising the target-sequence of the guide-polynucleotide and the plasmid are introduced into the cell either simultaneously or consecutively with at the least one of the at least two double-stranded polynucleotides.

In the method according to the invention, the assembly of the single-stranded or double-stranded oligonucleotide comprising the target-sequence of the guide-polynucleotide into the plasmid and the assembly of at least two double-stranded polynucleotides into a single double-stranded polynucleotide construct may occur essentially simultaneously within the cell.

In a second aspect, there is provided for a composition comprising the cell and at least one of at least two double-stranded polynucleotides as defined in the first aspect and further comprising the vector, preferably a plasmid, expressing the guide-polynucleotide as defined in the first aspect. The composition is herein referred to as a composition according to the invention

In this aspect, all features are preferably those of the first aspect of the invention.

In a third aspect, there is provided for a cell an assembled double-stranded polynucleotide construct, obtainable by or obtained by the method according to the first aspect of the invention. In this aspect, all features are preferably those of the first aspect of the invention.

The cell according to the third aspect of the invention, may further comprise a polynucleotide encoding a compound of interest. Said compound of interest may be any compound of interest and is preferably one as defined in the section “General Definitions”.

The cell according to the third aspect of the invention, may express a compound of interest. Said compound of interest may be native or may be foreign to the cell.

In a fourth aspect, there is provided for a method for the production of a compound of interest, comprising culturing the cell according to the third aspect of the invention under conditions conducive to the production of the compound of interest, and, optionally, purifying or isolating the compound of interest. In this aspect, all features are preferably those of the first, second and third aspect of the invention.

In a fifth aspect, there is provided for the use of a plurality of double-stranded polynucleotides in genome editing, wherein parts of the members of the plurality of double-stranded polynucleotides have sequence identity with parts of other members such that they can, within a cell, assemble into a double-stranded polynucleotide construct and wherein the double-stranded polynucleotide construct(s) can integrate into the genome of the cell within the proximity of a break into the genome. In this aspect, all features are preferably those of the first aspect of the invention.

### EMBODIMENTS

The following embodiments of the invention are provided; the features in these embodiments are preferably those as defined previously herein.

1. A method for genome editing within a cell comprising,

contacting the cell with at least two double-stranded polynucleotides such that the at least two double-stranded polynucleotides are introduced into the cell,

wherein a part of the first of the at least two double-stranded polynucleotides has sequence identity with a part of the second of the at least two double-stranded polynucleotides, such that, within the cell, the at least two double-stranded polynucleotides can assemble into a double-stranded polynucleotide construct,

wherein the double-stranded polynucleotide construct has at its 5′-end sequence identity with the genome of the cell within the proximity of a break in the genome of the cell and wherein the double-stranded polynucleotide construct has at its 3′-end sequence identity with the genome of the cell within the proximity of a break in the genome of the cell, and

wherein the double-stranded polynucleotide construct integrates into the genome of the cell within the proximity of the break in the genome of the cell.

2. A method according to embodiment 1, wherein at least three double-stranded polynucleotides are introduced into the cell,

wherein a part of the first of the at least three double-stranded polynucleotides has sequence identity with a part of the second of the at least three double-stranded polynucleotides and wherein a part of the second of the at least three double-stranded polynucleotides has sequence identity with the third of the at least three double-stranded polynucleotides, such that, within the cell, the at least three double-stranded polynucleotides can assemble into a double-stranded polynucleotide construct,

wherein the double-stranded polynucleotide construct has at its 5′-end sequence identity with the genome of the cell within the proximity of the break in the genome of the cell and wherein the double-stranded polynucleotide construct has at its 3′-end sequence identity with the genome of the cell within the proximity of the break in the genome of the cell, and

wherein the double-stranded polynucleotide construct integrates into the genome of the cell within the proximity of the break in the genome of the cell.

3. A method according to embodiment 2, wherein at least four, such as five, six, seven, eight, nine, ten, eleven, twelve, thirteen, fourteen, fifteen, sixteen, seventeen or eighteen double-stranded polynucleotides are introduced into the cell that are capable of assembly into the double-stranded polynucleotide construct.

4. A method according to any one of embodiments 1 to 3, wherein the double-stranded polynucleotide construct comprises at least one, such as two, three, four, five, six, seven or eight coding sequences.

5. A method according to any one of embodiments 1 to 4, wherein a plurality of double-stranded polynucleotides is introduced into the cell and assembles in a library of distinct double-stranded polynucleotide constructs, wherein each double-stranded polynucleotide construct is assembled from at least two double-stranded polynucleotides.

6. A method according to embodiment 5, wherein the library comprises at least two, such as three, four, five, six, seven or more distinct double-stranded polynucleotide constructs.

7. A method according to embodiment 5 or 6, wherein the distinct double-stranded polynucleotide constructs each integrate into a distinct locus in the genome of the cell.

8. A method according to any one of the preceding embodiments, wherein at least one of the double-stranded polynucleotide constructs comprises a marker.

9. A method according to any one of the preceding embodiments, wherein the method is performed iteratively, wherein in its iteration one or more distinct double-stranded polynucleotide constructs are assembled and integrated into the genome of the cell.

10. A method according to any one of the preceding embodiments, wherein the cell is a eukaryotic cell, such as a fungal cell, such as a non-conventional yeast cell, such as a Yarrowia cell, such as a Yarrowia lipolytica cell.

11. A method according to any one of the preceding embodiments, wherein the cell is deficient in an NHEJ (non-homologous end joining) component.

12. A method according to any one of the preceding embodiments, wherein the break is one selected from the group consisting of a single-stranded break (nick), an induced single-stranded break, a double-stranded break and an induced double-stranded break.

13. A method according to any one the preceding embodiments, wherein the break is induced by a functional genome editing system, preferably TALENs, CRISPR/Cas, CRISPR/Cpf1, or I-Scel.

14. A method according to any one of the preceding embodiments, wherein the cell expresses a functional heterologous genome editing enzyme, preferably a Cas enzyme, preferably Cas9 or Cas9 nickase; Cpf1; I-Scel, or wherein in the cell a heterologous genome editing enzyme, preferably a Cas enzyme, preferably Cas9 or Cas9 nickase; Cpf1; I-Scel, is present.

15. A method according to any one of the preceding embodiments, wherein a guide-polynucleotide is present within the cell.

16. A method according to any one of the preceding embodiments, wherein the guide-polynucleotide in the cell is expressed from a vector, preferably a plasmid, preferably the vector is introduced into the cell together with at least one of the at least two double-stranded polynucleotides.

17. A method according to embodiment 16, wherein the plasmid from which the guide-polynucleotide is expressed, is assembled within the cell by integration of a single-stranded or double-stranded oligonucleotide comprising the target-sequence of the guide-polynucleotide into the plasmid, wherein the single-stranded or double-stranded oligonucleotide comprising the target-sequence of the guide-polynucleotide and the plasmid are introduced into the cell either simultaneously or consecutively with at the least one of the at least two double-stranded polynucleotides.

18. A method according to embodiment 17, wherein the assembly of the single-stranded or double-stranded oligonucleotide comprising the target-sequence of the guide-polynucleotide into the plasmid and the assembly of at least two double-stranded polynucleotides into a single double-stranded polynucleotide construct occur essentially simultaneously within the cell.

19. A composition comprising the cell and at least one of at least two double-stranded polynucleotides as defined in any one of the preceding embodiments and further comprising the vector, preferably a plasmid, as defined in embodiment 16 or 17.

20. A cell comprising an assembled double-stranded polynucleotide construct, obtainable by the method according to any one of embodiments 1 to 18.

21. A cell obtainable by or produced by a method according to any one of embodiments 1 to 18, or the cell according to embodiment 20, further comprising a polynucleotide encoding a compound of interest.

22. A cell according to embodiment 21, expressing the compound of interest.

23. A cell according to embodiment 21 or 22, wherein the compound of interest is foreign to the cell.

24. A method for the production of a compound of interest, comprising culturing the cell according to any one of embodiments 21 to 23 under conditions conducive to the production of the compound of interest, and, optionally, purifying or isolating the compound of interest.

25. Use of a plurality of double-stranded polynucleotides in genome editing, wherein parts of the members of the plurality of double-stranded polynucleotides have sequence identity with parts of other members such that they can, within a cell, assemble into a double-stranded polynucleotide construct and wherein the double-stranded polynucleotide construct(s) can integrate into the genome of the cell within the proximity of a break into the genome.

### General Definitions

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element or more than one element.

The word “about” or “approximately” when used in association with a numerical value (e.g. about 10) preferably means that the value may be the given value (of 10) more or less 1% of the value.

Cas9, the single protein component in the class 2 type II-a CRISPR/Cas system (Mohanraju et al., 2016), is capable of complexing with two small RNAs named CRISPR RNA (crRNA) and transactivating crRNA (tracrRNA) to form a sequence-specific RNA-guided endonuclease (RGEN) whose target specificity is readily reprogrammed by either modifying the crRNA or using a single-chain guide RNA (sgRNA) composed of essential portions of crRNA and tracrRNA (Jinek et al., 2012). Cas9 RGENs cleave chromosomal DNA to produce site-specific DNA double-strand blunt-end breaks (DSBs) that are repaired by homologous recombination (HR) or non-homologous end-joining (NHEJ) to yield genetic modifications (Sander and Joung, 2014).

Cpf1 is a novel class 2 type V-a CRISPR RNA guided nuclease (Zetsche et al., 2015; Mohanraju et al., 2016). Cpf1 is different compared to Cas9 in various ways. Cpf1 is a single-RNA-guided nuclease and does not require a transactivating CRISPR RNA (tracrRNA), thus gRNAs are shorter in length than those for Cas9 by about 50%. Cpf1 cleavage produces cohesive (not blunt) double-stranded DNA breaks leaving 4-5-nt overhanging “sticky” ends, which might facilitate NHEJ-mediated transgene knock-in at target sites. Cpf1 recognizes thymidine-rich DNA PAM sequences, for example, 5′-TTTN-3′ or 5′-TTN-3′, which are located at the 5′ end of target-sequences (Zetsche et al., 2015) while Cas9 recognizes guanine-rich (NGG) PAMs located at the 3′-end of the target-sequence (Jinek et al., 2012).

Cpf1 is found in various bacteria including Francisella, Acidaminococcus and Lachnospiraceae (Zetsche et al., 2015). Heterologous Cpf1 RGEN activity was demonstrated in mammalian cells (Zetsche et al., 2015; Kim D. et al., 2015), mice (Kim, Y. et al., 2016, Hur et al., 2016), Drosophila (Port and Bullock, 2016) and rice plant (Xu et al., 2016).

A preferred nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

A further preferred backbone comprises a morpholino nucleotide analog or equivalent, in which the ribose ordeoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3-alkylene phosphonate, 5-alkylene phosphonate and chiral phosphonate, phosphinate, phosphoramidate including 3-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-S-, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the present invention of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Within the present invention, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLASTX program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gin or his; Asp to glu; Cys to ser or ala; Gin to asn; Glu to asp; Gly to pro; His to asn or gin; lie to leu or val; Leu to ile or val; Lys to arg; gin or glu; Met to leu or ile; Phe to met, leu or tyr; Ser to thr; Thr to ser; Trp to tyr; Tyr to trp or phe; and, Val to ile or leu.

A polynucleotide according to the present invention is represented by a nucleotide sequence. A polypeptide according to the present invention is represented by an amino acid sequence. A polynucleotide construct according to the present invention is defined as a polynucleotide which is isolated from a naturally occurring gene or which has been modified to contain segments of polynucleotides which are combined orjuxtaposed in a manner which would not otherwise exist in nature. Optionally, a polynucleotide present in a polynucleotide construct according to the present invention is operably linked to one or more control sequences, which direct the production or expression of the encoded product in a host cell or in a cell-free system.

The sequence information as provided herein should not be so narrowly construed as to require inclusion of erroneously identified bases. The skilled person is capable of identifying such erroneously identified bases and knows how to correct for such errors.

All embodiments of the present invention, preferably refer to a cell, not to a cell-free in vitro system; in other words, the systems according to the invention are preferably cell systems, not cell-free in vitro systems.

In all embodiments of the present invention, e.g., the cell according to the present invention may be a haploid, diploid or polyploid cell.

A cell according to the invention is interchangeably herein referred as “a cell”, “a cell according to the invention”, “a host cell”, and as “a host cell according to the invention”; said cell may be any cell, preferably a fungus, i.e. a yeast cell or a filamentous fungus cell, or it may be an algae, a microalgae or a marine eukaryote, e.g. a Labyrinthulomycetes host cell. Preferably, the cell is deficient in an NHEJ (non-homologous end joining) component. Said component associated with NHEJ is preferably a yeast Ku70, Ku80, MRE11, RAD50, RAD51, RAD52, XRS2, SIR4, LIF1, NEJ1 and/or LIG4 or homologue thereof. Alternatively, in the cell according to the invention NHEJ may be rendered deficient by use of a compound that inhibits RNA ligase IV, such as SCR7 (Vartak S V and Raghavan, 2015). The person skilled in the art knows how to modulate NHEJ and its effect on RNA-guided nuclease systems, see e.g. WO2014130955A1; Chu et al., 2015; et al., 2015; Song et al., 2015 and Yu et al., 2015; all are herein incorporated by reference. The term “deficiency” is defined elsewhere herein.

When the cell according to the invention is a yeast cell, a preferred yeast cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyceslactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica ATCC18943, Yarrowia lipolytica CLIB122, Pichia stipidis and Pichia pastoris.

The host cell according to the present invention is a filamentous fungal host cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK).

The filamentous fungal host cell may be a cell of any filamentous form of the taxon Trichocomaceae (as defined by Houbraken and Samson in Studies in Mycology 70: 1-51.2011). In another preferred embodiment, the filamentous fungal host cell may be a cell of any filamentous form of any of the three families Aspergillaceae, Thermoascaceae and Trichocomaceae, which are accommodated in the taxon Trichocomaceae.

The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell according to the present invention is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255(ATCC28089); even more preferably the filamentous fungal host cell according to the present invention is an Aspergillus niger. When the host cell according to the present invention is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells according to the present invention are Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Rasamsonia emersonii CBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

Preferably, and more preferably when the microbial host cell according to the invention is a filamentous fungal host cell, a host cell according to the present invention further comprises one or more modifications in its genome such that the host cell is deficient in the production of at least one product selected from glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE if compared to a parent host cell and measured under the same conditions. A modification, preferably in the genome, is construed herein as one or more modifications. A modification, preferably in the genome of a host cell according to the present invention, can either be effected by
a) subjecting a parent host cell to recombinant genetic manipulation techniques; and/or
b) subjecting a parent host cell to (classical) mutagenesis; and/or
c) subjecting a parent host cell to an inhibiting compound or composition. Modification of a genome of a host cell is herein defined as any event resulting in a change in a polynucleotide sequence in the genome of the host cell.

Preferably, a host cell according to the present invention has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes for a partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In one embodiment the mutant microbial host cell according to the invention comprises a modification in its genome selected from
a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32, (7) electronic access http://nar.oupjournals.org/cgi/reprint/32/7/e59 or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene replacement or partial or complete gene deletion.

For example, in case of replacement of a polynucleotide, polynucleotide construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example a polynucleotide corresponding to the endogenous polynucleotide may be replaced by a defective polynucleotide; that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified.

Alternatively or in combination with other mentioned techniques, a technique based on recombination of cosmids in an E. coli cell can be used, as described in: A rapid method for efficient gene replacement in the filamentous fungus Aspergillus nidulans (2000) Chaveroche, M-K., Ghico, J-M. and d'Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52).

A modification resulting in reduced or no production of undesired compound is preferably due to a reduced production of the mRNA encoding said undesired compound if compared with a parent microbial host cell which has not been modified and when measured under the same conditions.

A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in WO2008/053019, WO02005/05672A1, WO02005/026356A1, Oliveira et al.; Crook et al., 2014; and/or Barnes et al., may be used at this purpose.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath_et al, (2003) Nature: Systematic functional analysis of the Caenorhabditis elegans genome using RNAi. Vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez 0, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6) or by the use of inhibitors inhibiting enzymatic activity of an undesired polypeptide as described herein (e.g. nojirimycin, which function as inhibitor for β-glucosidases (Carrel F.L.Y. and Canevascini G. Canadian Journal of Microbiology (1991) 37(6): 459-464; Reese E. T., Parrish F. W. and Ettlinger M. Carbohydrate Research (1971) 381-388)).

In an embodiment of the present invention, the modification in the genome of the host cell according to the invention is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell according to the present invention produces 1% less of the un-desired compound if compared with the parent host cell which has not been modified and measured under the same conditions, at least 5% less of the un-desired compound, at least 10% less of the un-desired compound, at least 20% less of the un-desired compound, at least 30% less of the un-desired compound, at least 40% less of the un-desired compound, at least 50% less of the un-desired compound, at least 60% less of the un-desired compound, at least 70% less of the un-desired compound, at least 80% less of the un-desired compound, at least 90% less of the un-desired compound, at least 91% less of the un-desired compound, at least 92% less of the un-desired compound, at least 93% less of the un-desired compound, at least 94% less of the un-desired compound, at least 95% less of the un-desired compound, at least 96% less of the un-desired compound, at least 97% less of the un-desired compound, at least 98% less of the un-desired compound, at least 99% less of the un-desired compound, at least 99.9% less of the un-desired compound, or most preferably 100% less of the un-desired compound.

A reference herein to a patent document or other matter which is given as prior art is not to be taken as an admission that that document or matter was known or that the information it contains was part of the common general knowledge as at the priority date of any of the claims.

The sequence information as provided herein should not be so narrowly construed as to require inclusion of erroneously identified bases. The skilled person is capable of identifying such erroneously identified bases and knows how to correct for such errors.

The disclosure of each reference set forth herein is incorporated herein by reference in its entirety. The present invention is further illustrated by the following examples:

### EXAMPLES

In the following Examples, various embodiments of the invention are illustrated. From the above description and these Examples, one skilled in the art can make various changes and modifications of the disclosure to adapt it to various usages and conditions.

### Example 1: Efficient Genome Editing of Yarrowia lipolytica Mediated by CRISPR-Cas9 Using 50-Bp Homology Sequences for In Vivo Assembly and Targeted Integration

This example describes the efficient integration of donor DNA, a GFP expression cassette, into the INT05 integration locus of Yarrowia lipolytica strain ML3243. Yarrowia strain ML3243 was transformed with donor DNA and plasmid pSTV086 harboring a Cas9 expression cassette as well as a guide RNA expression cassette for targeting Cas9 to the INT05 locus in the genome. The donor DNA that was used in transformation was either a single fragment (SEQ ID NO: 4) or 2 bipartite fragments (5′ fragment, SEQ ID NO: 5 and 3′ fragment, SEQ ID NO: 6). In case the donor DNA was a single fragment, the fragment comprises INT05 genomic DNA 5′ flank 50-bp, the YI_HSP promoter, GFP expression ORF and YI_GPD terminator and 3′ flank 50-bp INT05 genomic DNA (SEQ ID NO:4). In case the donor DNA consisted of 2 bipartite fragments, the 5′ fragment comprised the INT05 genomic DNA 5′ flank 50-bp, the YI_HSP promoter, truncated GFP expression ORF (SEQ ID NO: 5) and the 3′ fragment comprised the 3′ part of the GFP ORF, YI_GPD terminator and INT05 genomic DNA 3′ flank sequence (SEQ ID NO: 6). For assembly of a functional GFP gene, both bipartite fragments had an overlap of 50-bp in the GFP ORF. Incorporation of the GFP expression cassette assembled from the two bipartite donor DNA fragments, or as a single donor DNA fragment, results in a fluorescent strain after transformation. Transformants were selected on YEPD agar plates that are supplemented with 150 ug/ml hygromycin B (pSTV086 confers resistance to hygB) and fluorescence of the strains was determined.

Construction of Cas9-Expressinq Yarrowia lipolytica Strain ML3243 ((MATa ΔKU70 Cas9)

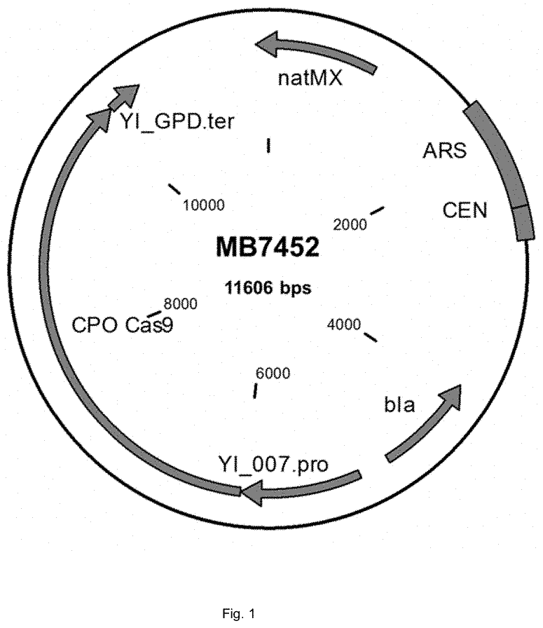

The Yarrowia plasmid for expression of Cas9, MB7452 (FIG. 1, SEQ ID NO: 2), was transferred to Yarrowia strain ML324 (MATa; deposited under number ATCC18943). Yarrowia vector MB7452 contains a Cas9 expression cassette consisting of a codon optimized Cas9 gene expressed from the YI_007 promoter (Yarrowia lipolytica promoter of YALIOB14377g), the YI_GPD terminator (Yarrowia lipolytica terminator of YALI0C06369g), and a functional NatMX marker cassette conferring resistance against nourseothricin.

Vector MB7452 containing the Cas9 expression cassette was transformed to Yarrowia lipolytica strain ML324 (MATa) using the LiAc/salmon sperm (SS) carrier DNA/PEG method (Gietz and Woods, 2002) with a heat shock temperature of 39 degrees Celsius. In the transformation mixture one microgram of vector MB7452 was used. The transformation mixture was plated on YPD-agar (10 grams per liter of yeast extract, 20 grams per liter of peptone, 20 grams per liter of dextrose, 20 grams per liter of agar) containing 150 microgram (μg) nourseothricin (NTC, Jena Bioscience, Germany) per ml. After two to four days of culture at 30 degrees, Celsius transformants appeared on the transformation plate. A transformant conferring resistance to nourseothricin on the plate, designated strain ML3242 (MATa, Cas9), was inoculated in YPD-nourseothricin medium (10 grams per liter of yeast extract, 20 grams per liter of peptone, 20 grams per liter of dextrose, 150 μg nourseothricin (NTC, Jena Bioscience, Germany) per ml), and used in a subsequent transformation to knock out the KU70 gene.

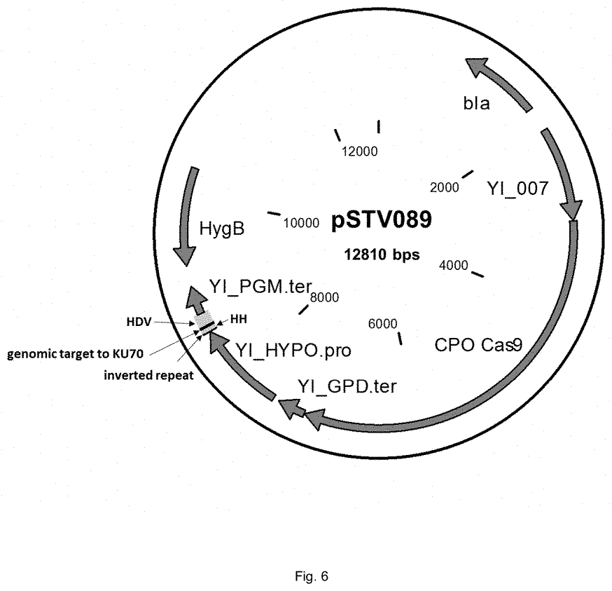

The CRISPR/Cas mediated knockout of the KU70 gene in Yarrowia strain ML3242 was performed by transformation of plasmid pSTV089 and a 100-bp KU70 knock out donor DNA fragment to the strain. Yarrowia plasmid pSTV089 (SEQ ID NO:56, FIG. 6) is equipped with a guide-RNA expression cassette and a functional HygB marker cassette conferring resistance to hygromycin B. The guide-RNA expression cassette targets the KU70 gene in the Yarrowia genome and is comprised of the YI_HYPO promoter (SEQ ID NO: 12) followed by a 6 bp inverted repeat of the KU70 genomic target (SEQ ID NO: 57), a hammerhead (HH) (SEQ ID NO: 10) and HDV ribozyme (SEQ ID NO: 11) on the 5′ and 3′ side of the 20 bp genomic target-sequence of KU70 (SEQ ID NO: 58) and the YI_PGM terminator (SEQ ID NO: 14), as described by Gao and Zhao, 2014. In addition to the guide-RNA expression cassette and HygB marker cassette, plasmid pSTV089 contains a Cas9 expression cassette. Cas9 was codon optimized for expression in Y. lipolytica and was expressed from the Yarrowia lipolytica 007 promoter (SEQ ID NO: 15) and the Yarrowia lipolytica GPD terminator (SEQ ID NO: 13). The 100-bp KU70 knock out donor DNA fragment (SEQ ID NO: 59) is a double stranded DNA fragment and comprises 50-bp upstream and 50-bp downstream of the KU70 gene. Upon incorporation of the KU70 knock out donor DNA fragment the KU70 gene that is in between the 50-bp sequences was deleted from the genome.

Plasmid pSTV089 and the donor DNA fragment were transformed to Yarrowia lipolytica strain ML3242 (MATa Cas9) using the LiAc/salmon sperm (SS) carrier DNA/PEG method (Gietz and Woods, 2002) with a heat shock temperature of 39 degrees Celsius. In the transformation mixture 500 nanogram of plasmid pSTV089 was used and 500 ng of the 100-bp KU70 knock out donor DNA fragment. The transformation mixture was plated on YPD-agar (10 grams per liter of yeast extract, 20 grams per liter of peptone, 20 grams per liter of dextrose, 20 grams per liter of agar) containing 150 microgram (μg) hygromycin B (Thermo Fisher Scientific, The Netherlands, Cat no: 10687010) per ml and 150 microgram (μg) nourseothricin (NTC, Jena Bioscience, Germany) per ml. After two to four days of culture at 30 degrees Celsius, transformants appeared on the transformation plate. Transformants were selected for presence of the Cas9 expression plasmid (MB7452) by nourseothricin resistance and presence of plasmid pSTV089 by hygromycin B resistance.

The knock out of the KU70 gene was confirmed by PCR. As template, genomic DNA isolated using the YeaStar genomic DNA kit (D2002, ZymoResearch, BaseClear, The Netherlands) according to supplier's manual, was used. Primer set (SEQ ID NO: 60 and SEQ ID NO: 61), located on the genome just outside the 50-bp sequences upstream and downstream of the KU70 gene used for the knock out, was used with PrimeStar polymerase according to supplier's manual. The knock out was confirmed by amplification of a 964-bp fragment that confirms deletion of the KU70 gene and integration of the KU70 knock out donor DNA.

Since an ML3242 transformant in which the KU70 knock out was confirmed by PCR was to be used in additional Cas9 experiments, it was cured from plasmid pSTV089 (hygromycin B marker) while maintaining its Cas9 expression plasmid, MB7452 (nourseothricin marker). The strain was cultured for 24 hours in YPD liquid medium (10 grams per liter of yeast extract, 20 grams per liter of peptone, 20 grams per liter of dextrose) supplemented with 150 microgram (μg) nourseothricin (NTC, Jena Bioscience, Germany) per ml at 30° C., shaking speed: 250 rpm. Dilutions of the culture were made in milliQ and subsequently plated onto YPD-agar medium (10 grams per liter of yeast extract, 20 grams per liter of peptone, 20 grams per liter of dextrose, 20 grams per liter of agar) containing 150 microgram (μg) nourseothricin (NTC, Jena Bioscience, Germany) per ml. After two to four days of growth at 30° C., colonies appeared on the agar plate. Single colonies were subsequently checked for hygromycin B sensitivity by streaking them on YPD-agar (10 grams per liter of yeast extract, 20 grams per liter of peptone, 20 grams per liter of dextrose, 20 grams per liter of agar) containing 150 microgram (μg) hygromycin B (Thermo Fisher Scientific, The Netherlands, Cat no: 10687010) per ml. A hygromycin B sensitive strain was selected and designated ML3243 (MATa ΔKU70 Cas9). This strain was used in further transformation experiments.

Double-Stranded DNA (Ds-DNA) GFP Donor DNA Cassette

A double-stranded donor DNA expression cassette coding for the green fluorescent protein, Aequorea victoria eGFP (A. vic_eGFP), with 50-bp genomic flanks on either side for integration into the INT05 locus of the genome of Y. lipolytica strain ML3243, was ordered as synthetic DNA at Baseclear (Leiden, The Netherlands). The eGFP expression is controlled by the YI_HSP promoter (Yarrowia promotor of YALIOD20526g, SEQ ID NO: 16) and YI_GPD terminator (Yarrowia terminator of YALI0C06369g, SEQ ID NO: 13). The full sequence of the donor DNA fragment is presented in SEQ ID NO: 4. PCR reactions using primer set (SEQ ID NO: 17 and SEQ ID NO: 18) were performed to obtain donor DNA fragments in higher quantities that are later to be used in the transformation experiments.

Bi-Partite Double-Stranded DNA (Ds-DNA) GFP Donor DNA Cassettes

Bipartite donor DNA fragments for expression of the green fluorescent protein, Aequorea victoria eGFP (A. vic_eGFP) were created by PCR. As template in the PCR reaction the single donor DNA expression cassette (SEQ ID NO:4) as described above was used. The bipartite fragments were obtained by PCR reactions, the 5′ bipartite fragment was amplified by primer set (SEQ ID NO: 19 and SEQ ID NO: 20) and the 3′ bipartite fragment was amplified by primer set (SEQ ID NO: 21 and SEQ ID NO: 22). The 5′ bipartite fragment comprises the 5′ genomic DNA INT05 flank 50-bp —YI_HSP promoter-truncated A. vic_eGFP (25 bp) and is in total 1075 bp. The 3′ bipartite fragment comprises the YI_HSP promoter (25 bp)—A.vic_eGFP-YI.GPD terminator-3′ genomic DNA INT05 flank 50-bp and is in total 1117 bp. For in vivo assembly, the last 50-bp of the 5′ bipartite fragment are identical to the first 50-bp of the 3′ bipartite fragment.

Integration Site INT05

The INT05 integration site is a non-coding region between gene YALI0F11275g and YAL10F11297g, located on chromosome NC_006072.

pSTV086 Vector (Yarrowia Expression Vector. HyqB Marker)

Yarrowia vector pSTV086 (SEQ ID NO: 3) is equipped with a guide-RNA expression cassette and a functional HygB marker cassette conferring resistance to hygromycin B. The guide-RNA expression cassette targets the INT05 integration site in the Yarrowia genome and is comprised of the YI_HYPO promoter followed by a 6 bp inverted repeat of the genomic target, a hammerhead (HH) and hepatitis delta virus (HDV) ribozyme on the 5′ and 3′ side of the 20 bp genomic target-sequence of INT05, as described by Gao and Zhao, 2014 and the YI_PGM terminator.

The parts of the guide-RNA expression cassette are set out as follows: SEQ ID NO: 7 represents the genomic target-sequence of INT05, SEQ ID NO: 9 represents the 6 bp inverted repeat of the INT05 genomic target, SEQ ID NO: 10 and SEQ ID NO: 11 represent the HH ribozyme and HDV ribozyme respectively, SEQ ID NO: 12 represents the YI_HYPO promoter and SEQ ID NO: 14 represent the YI_PGM terminator.

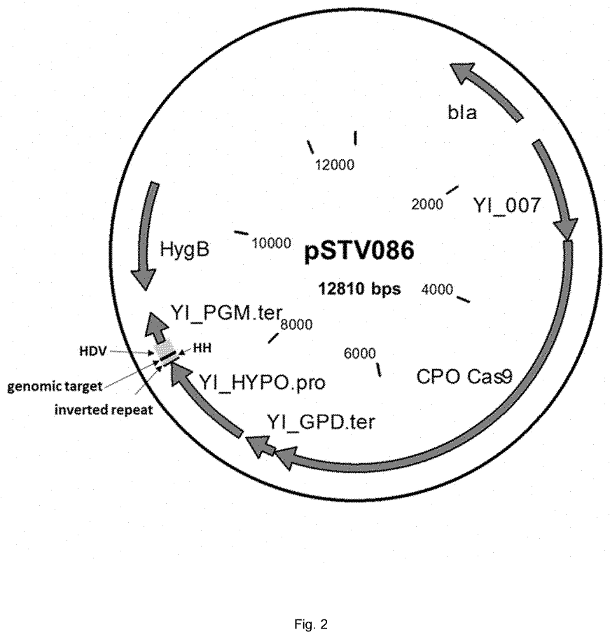

In addition to the guide-RNA expression cassette and HygB marker cassette, plasmid pSTV086 contains a Cas9 expression cassette. Cas9 is codon optimized for expression in Y. lipolytica and is expressed from the Yarrowia lipolytica 007 promoter and the Yarrowia lipolytica GPD terminator. A plasmid map of plasmid pSTV086 is depicted in FIG. 2.

DNA Concentrations

All DNA concentrations, including the donor DNA fragments and plasmid pSTV086, were determined using a NanoDrop device (ThermoFisher, Life Technologies, Bleiswijk, the Netherlands), providing the concentrations in nanogram per microliter. Based on these measurements, an amount of 500 ng pSTV086 plasmid and 500 ng donor DNA fragment(s) were used in the transformation experiments.

PCR Reactions

The PrimeSTAR GXL DNA polymerase (TaKaRa, supplied by VWR, Amsterdam Leiden, the Netherlands. Cat no. R050A) was used in the PCR reactions described above. PCR reactions were performed according to manufacturer's instructions.

PCR Purification

Purification of PCR reactions was performed using NucleoSpin Gel and PCR Clean-up kit (Machery-Nagel, distributed by Bioke, Leiden, the Netherlands) according to manufacturer's instructions.

Yarrowia Transformation

Strain pML3243 expressing Cas9 was inoculated in YPD-nourseothricin medium (10 grams per liter of yeast extract, 20 grams per liter of peptone, 20 grams per liter of dextrose, 150 μg nourseothricin (NTC, Jena Bioscience, Germany) per ml). Subsequently, strain ML3243 was transformed with 500 ng pSTV086 plasmid, 500 ng donor DNA fragment (single fragment, size: 2142 bp) or bipartite fragments; 5′ fragment: 500 ng (size: 1076 bp) and 3′ fragment: 500 ng (size: 1117 bp), as indicated in Table 1, using the LiAc/SS carrier DNA/PEG method (Gietz and Woods, 2002) with a heat shock temperature of 39 degrees Celsius.

TABLE 1
Overview of DNA's used in the transformation
round of Y. lipolytica strain ML3243.

| | Donor DNA 5′ | Donor DNA 3′ | Donor DNA | |
|---|---|---|---|---|
| | bipartite | bipartite | Single | |
| Transfor- | fragment | fragment | fragment | Plasmid |
| mation | (500 ng) | (500 ng) | (500 ng) | (500 ng) |
| #1 | INT05 - | 3′ part GFP | | pSTV086 |
| | 5′ part GFP | cassette - | | |
| | cassette | INT05 | | |
| | SEQ ID NO: 5 | SEQ ID NO: 6 | | |
| #2 | — | — | INT05 - GFP | pSTV086 |
| | | | cassette - | |
| | | | INT05 | |
| | | | SEQ ID NO: 4 | |
| #3 | — | — | — | pSTV086 |
| (control) | | | | |
| #4 | — | — | — | — |
| (no DNA | | | | |
| control) | | | | |

The transformation mixtures were plated on YPD-agar (10 grams per liter of yeast extract, 20 grams per liter of peptone, 20 grams per liter of dextrose, 20 grams per liter of agar) containing 150 μg hygromycin B (Thermo Fisher Scientific, the Netherlands) per ml. The plates were incubated at 30 degrees Celsius until colonies appeared on the plates.

Transformants were checked for incorporation of the donor DNA, being a GFP expression cassette. Fluorescence of the strains was visualized by the QPix450 (Molecular Devices, Filter: Ex/Em: 457/536 nm—FITC/GFP). The success rate of GFP integration on INT05 locus based on phenotype is summarized below in Table 2.

TABLE 2
Overview of the total number and number of fluorescent
transformants obtained after transformation of pSTV086 and donor
DNA fragment (INT05 flank (50-bp) - GFP expression cassette -
INT05 flank (50-bp)) or bipartite donor DNA fragments (INT05
flank (50-bp) - truncated GFP expression cassette and trunctated
GFP expression cassette - INT05 flank (50-bp). The number of
colonies result from plating the transformation mixtures on
YPD-agar plates supplemented with 150 μg/ml hygromycin B.
Percentages wee calculated by dividing the number of colonies
by the total amount of colonies of that same transformation.

| | | | Number of non |
|---|---|---|---|
| | | Number of green | fluorescent |
| Transfor- | Total number of | fluorescent (GFP) | transformants |
| mation | transformants | transformants | (white) |
| #1 | 393 (100%) | 294 (75%) | 99 (25%) |
| #2 | 378 (100%) | 287 (76%) | 91 (24%) |
| #3 | 71 (100%) | — | 71 (100%) |
| (control) | | | |
| #4 | 0 | — | — |
| (no DNA | | | |
| control) | | | |

In this example, correct in vivo assembly of bi-partite fragments, with 50-bp overlap, into a functional GFP expression cassette has been demonstrated. Correct targeting of the donor DNA into the INT05 locus of the Yarrowia genome using 50-bp genomic DNA flanks was checked by PCR. As template, genomic DNA of fluorescent transformants, was used. Genomic DNA was isolated using the YeaStar genomic DNA kit (D2002, ZymoResearch, BaseClear, The Netherlands) according to supplier's manual. Using primer set (SEQ ID NO: 43 and SEQ ID NO: 44), that is located on the genome just outside the INT05 50-bp flanks that were used for integration, and PrimeStar polymerase according to supplier's manual, a 3.5 kb fragment was amplified that confirms correct targeted integration of the donor DNA at the INT05 locus.

### Example 2: Efficient In Vivo Assembly of 3 Expression Cassettes and Targeted Integration into the Genome of Yarrowia lipolytica, Mediated by CRISPR-Cas9

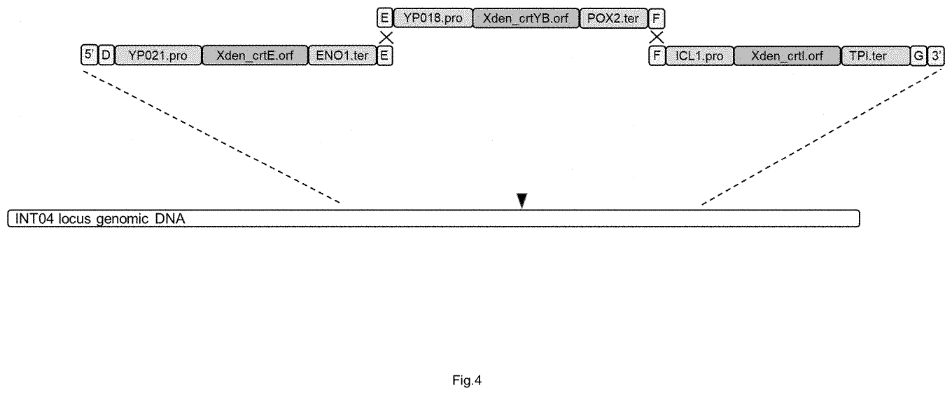

This example describes the efficient in vivo assembly and integration of a 3-gene pathway (donor DNA fragments; X. dendrorhous crtE, crtYB and crtl expression cassettes codon optimized for expression in Y. lipolytica (SEQ ID NO: 23, 24, 25)), into the INT04 integration locus of Yarrowia lipolytica strain ML3243. Yarrowia strain ML3243 is transformed with 3 donor DNA fragments and plasmid pSTV085 harboring a guide RNA expression cassette targeting INT04 as well as a Cas9 expression cassette. The donor DNA fragments form a heterologous metabolic pathway of 3 genes (crtE, crtYB and crtl) involved in the biosynthesis of beta-carotene. Donor DNA cassettes contain 50-bp connector sequences to allow in vivo recombination into a single stretch of DNA, the outside cassettes contain 50-bp INT04 genomic DNA flanks on either the 5′ or 3′ side for targeted integration of the pathway genes as depicted in FIG. 4. Upon integration of all 3 donor DNA expression cassettes, carotenoids were produced resulting in yellow to orange colonies. Transformants were selected on YEPD agar plates that were supplemented with 150 ug/ml hygromycin B (pSTV085). Editing efficiency was calculated by counting the number of colored colonies, producing carotenoids divided by the total number of transformants on a transformation plate.

Yarrowia lipolytica Strain ML3243

Yarrowia lipolytica strain ML3243 is expressing Cas9. Construction of Y. lipolytica strain ML3243 is described in Example 1.

Integration Site INT04

The INT04 integration site is a non-coding region between gene YALI0E12133g and YALI0E12111g, located on chromosome E.

pSTV085 Vector (Yarrowia Expression Vector, HyqB Marker)

Yarrowia vector pSTV085 (SEQ ID NO:55) is equipped with a guide-RNA expression cassette and a functional HygB marker cassette conferring resistance to hygromycin B. The guide-RNA expression cassette targets the INT04 integration site in the Yarrowia genome and is comprised of the YI_HYPO promoter followed by a 6 bp inverted repeat of the genomic target, a hammerhead (HH) and HDV ribozyme on the 5′ and 3′ side of the 20 bp genomic target-sequence of INT04, as described by Gao and Zhao, and the YI_PGM terminator.

The parts of the guide-RNA expression cassette are set out in the following SEQ ID NO: 26 represents the genomic target-sequence of INT04, SEQ ID NO: 27 represents the 6 bp inverted repeat of the INT04 genomictarget, SEQ ID NO: 10 and SEQ ID NO: 11 represent the HH ribozyme and HDV ribozyme respectively, SEQ ID NO: 12 represents the YI_HYPO promoter and SEQ ID NO: 14 represent the YI_PGM terminator.

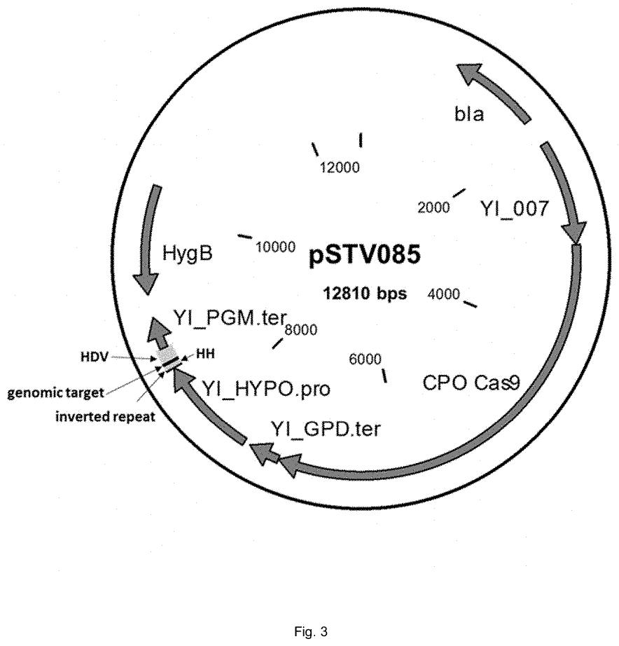

In addition to the guide-RNA expression cassette and HygB marker cassette, plasmid pSTV085 contains a Cas9 expression cassette. Cas9 is codon optimized for expression in Y. lipolytica and is expressed from the Yarrowia lipolytica 007 promoter and the Yarrowia lipolytica GPD terminator. A plasmid map of plasmid pSTV086 is depicted in FIG. 3.

Donor DNA—Carotenoid Expression Cassettes (Xanthophyllomyces Dendrorhous (Xden) crtE, crtYB and Crtl)

Donor DNA expression cassettes (crtE, crtYB, crt), codon optimized for expression in Yarrowia lipolytica, were synthesized at BaseClear (Leiden, The Netherlands). The crtE expression cassette contains a Y. lipolytica YPO21 (YI_YP021) promoter and an ENO1 (YI_ENO1) terminator sequence.

The crtYB expression cassette contains a Y. lipolytica YP018 (YI_YP018) promoter and a POX2 (YI_POX2) terminator sequence. The crtl expression cassette contains a Y. lipolytica ICL1 (YI_ICL1) promoter and a TPI (YI_TPI) terminator sequence. The donor DNA expression cassettes contain 50-bp DNA flank sequences for targeted integration in INT04 and/or 50-bp connector sequences to allow for in vivo recombination and integration into genomic DNA as one stretch of DNA, as depicted in FIG. 4.

In Table 3 an overview of the SEQ ID NO's corresponding to the genetic elements of the donor DNA (carotenoid expression cassettes) fragments is provided.

TABLE 3
Overview of SEQ ID NO's representing all genetic parts
from the carotenoid expression cassettes (donor DNA).

Donor DNA

| | |
|---|---|
| YI_INT04 FLANK5 - COND - YI_YP021.pro - | SEQ ID NO: 23 |
| Xden_crtE.orf - YI_ENO1.ter - CONE | |
| YI_INT04 FLANK5 | SEQ ID NO: 28 |
| COND | SEQ ID NO: 29 |
| YI_YP021.pro | SEQ ID NO: 30 |
| Xden_crtE.orf | SEQ ID NO: 31 |
| YI_ENO1.ter | SEQ ID NO: 32 |
| CONE | SEQ ID NO: 33 |
| CONE - YI_YP018.pro - Xden_crtYB.orf - | SEQ ID NO: 24 |
| YI_POX2.ter - CONF | |
| YI_YP018.pro | SEQ ID NO: 34 |
| Xden_crtYB.orf | SEQ ID NO: 35 |
| YI_POX2.ter | SEQ ID NO: 36 |
| CONF | SEQ ID NO: 37 |
| CONF - YI_ICL1.pro - Xden_crtl.orf - | SEQ ID NO: 25 |
| YI_TPI.ter - CONG - YI_INT04 FLANK3 | |
| YI_ICL1.pro | SEQ ID NO: 38 |
| Xden_crtl.orf | SEQ ID NO: 39 |
| YI_TPI.ter | SEQ ID NO: 40 |
| CONG | SEQ ID NO: 41 |
| YI_INT04 FLANK3 | SEQ ID NO: 42 |

DNA concentrations

All DNA concentrations, including the donor DNA fragments and plasmid pSTV085, were determined using a NanoDrop device (ThermoFisher, Life Technologies, Bleiswijk, the Netherlands), providing the concentrations in nanogram per microliter. Based on these measurements, an amount of 300 ng pSTV085 plasmid and 100 ng per kb donor DNA fragment(s) were used in the transformation experiments.

PCR reactions

PrimeSTAR GXL DNA polymerase (TaKaRa, supplied by VWR, Amsterdam Leiden, the Netherlands. Cat no. R050A) was used in the PCR reactions described above. PCR reactions were performed according to manufacturer's instructions.

PCR Purification

Purification of PCR reactions was performed using NucleoSpin Gel and PCR Clean-up kit (Machery-Nagel, distributed by Bioke, Leiden, the Netherlands) according to manufacturer's instructions.

Yarrowia Transformation

Strain ML3243, which is expressing Cas9, was inoculated in YPD-nourseothricin medium (10 grams per liter of yeast extract, 20 grams per liter of peptone, 20 grams per liter of dextrose, 150 μg nourseothricin (NTC, Jena Bioscience, Germany) per ml. Subsequently, strain ML3243 was transformed with 300 ng pSTV085 plasmid and the 3 donor DNA fragments that, after correct in vivo assembly into genomic DNA of the host, enable heterologous production of beta-carotene. The 3 donor DNA fragments, being the carotenoid expression cassettes, were added in equimolar amounts: 100 ng donor DNA per kb fragment, as indicated in Table 4. Transformation was performed according to the LiAc/SS carrier DNA/PEG method (Gietz and Woods, 2002) with a heat shock temperature of 39 degrees C.

TABLE 4
Overview of DNA's used in the transformation
round of Y. lipolytica strain ML3243.

| | | Size (bp) | | Plasmid |
|---|---|---|---|---|
| Transformation | Donor DNA | Amount (ng) | | (300 ng) |
| #1 | YI_INT04 FLANK5 - | 2192 | bp | pSTV085 |
| | COND - YI_YP021.pro - | 219 | ng | |
| | Xden_crtE.orf - | | | |
| | YI_ENO1.ter - CONE | | | |
| | CONE-YI_YP018.pro - | 3033 | bp | |
| | Xden_crtYB.orf - | 303 | ng | |
| | YI_POX2.ter - CONF | | | |
| | CONF - YI_ICL1.pro - | 3210 | bp | |
| | Xden_crtl.orf - | 321 | ng | |
| | YI_TPI.ter - CONG - | | | |
| | YI_INT04 FLANK3 | | | |

| | | | |
|---|---|---|---|
| #2 | — | — | pSTV085 |
| (control) | | | |
| #3 | — | — | — |
| (no DNA | | | |
| control) | | | |

The transformation mixtures were plated on YPD-agar (10 grams per liter of yeast extract, 20 grams per liter of peptone, 20 grams per liter of dextrose, 20 grams per liter of agar) containing 150 μg hygromycin B (Thermo Fisher Scientific, the Netherlands) per ml. The plates were incubated at 30 degrees Celsius until colonies appeared on the plates.

Transformants were checked for correct incorporation of all 3 donor DNA cassettes, resulting in beta carotene production which can be seen by orange colored colonies (orange phenotype). The orange color of transformants will only be visible when all 3 cassettes are present in the strain. The success rate of the integration of carotenoid genes on INT04 locus based on phenotype is summarized below in Table 5.

TABLE 5
Overview of the total number and number of
orange transformants obtained after transformation
of pSTV085 and donor DNA fragments.

| | Total number | Number of | Number |
|---|---|---|---|
| | of | orange | white |
| Transformation | transformants | transformants | transformants |
| #1 | 56 (100%) | 38 (68%) | 18 (32%) |
| #2 | 12 (100%) | — | 12 (100%) |
| (control) | | | |
| #3 | 0 | — | — |
| (no DNA | | | |
| control) | | | |

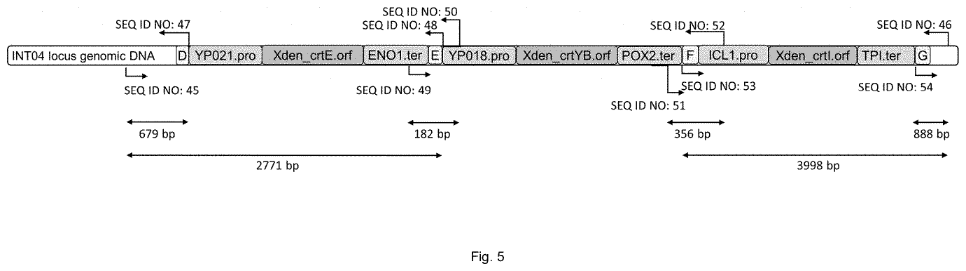

As a result of integration of all 3 donor DNA fragments, carotenoids were produced, resulting in colonies with an orange phenotype on plate. Correct assembly of the 3 genes as well as correct targeting on the INT04 locus was demonstrated by PCR, of which the set-up is displayed in FIG. 5. Genomic DNA of transformants with an orange phenotype is isolated using YeaStar genomic DNA kit (D2002, ZymoResearch, BaseClear, The Netherlands) according to supplier's manual. Using PrimeSTAR GXL DNA polymerase and the primersets as depicted in FIG. 5 and presented in Table 6, the correct integration on the INT04 locus as well as correct assembly of the carotenoid expression cassettes was confirmed.

TABLE 6
Overview of primersets used to confirm correct integration on
INT04 locus in the genome and assembly of the donor DNA fragments
(carotenoid expression cassettes) used in transformation.

| Fragment | Primer set | Size | |
|---|---|---|---|
| INT04 - COND | SEQ ID NO: 45 | 679 | bp |
| | SEQ ID NO: 47 | | |
| INT04 - CONE | SEQ ID NO: 45 | 2771 | bp |
| | SEQ ID NO: 48 | | |
| YI_ENO1.ter - YI_YP018.pro | SEQ ID NO: 49 | 182 | bp |
| | SEQ ID NO: 50 | | |
| YI_POX2.ter - YI_ICLI.pro | SEQ ID NO: 51 | 356 | bp |
| | SEQ ID NO: 52 | | |
| CONF - INT04 | SEQ ID NO: 53 | 3998 | bp |
| | SEQ ID NO: 46 | | |
| CONG - INT04 | SEQ ID NO: 54 | 888 | bp |
| | SEQ ID NO: 46 | | |

In this example targeted integration and correct in vivo assembly of the donor DNA fragments, comprising the carotenoid pathway, with 50-bp overlap has been demonstrated.

### REFERENCES

Gao and Zhao, 2014; Self-processing of ribozyme-flanked RNAs into guide RNAs in vitro and in vivo forCRISPR-mediated genome editing. J. Integrative Plant Biology, vol 56, 4, pages 343-349.
Mali et al., Science. 2013 Feb. 15; 339(6121):823-6
Cong et al., Science. 2013 Feb. 15; 339(6121):819-23
Zetsche et al., Cell. 2015 Oct. 22; 163(3):759-71
Gao et al, Integr Plant Biol. 2014 April; 56(4):343-9

1. A method for genome editing within a cell comprising,
contacting the cell with at least two double-stranded polynucleotides such that the at least two double-stranded polynucleotides are introduced into the cell,
wherein a part of the first of the at least two double-stranded polynucleotides has sequence identity with a part of the second of the at least two double-stranded polynucleotides, such that, within the cell, the at least two double-stranded polynucleotides can assemble into a double-stranded polynucleotide construct,
wherein the double-stranded polynucleotide construct has at a 5′-end thereof sequence identity with a genome of the cell within proximity of a break in the genome of the cell and wherein the double-stranded polynucleotide construct has at a 3′-end thereof sequence identity with a genome of the cell within proximity of a break in the genome of the cell, and
wherein the double-stranded polynucleotide construct integrates into the genome of the cell within the proximity of the break in the genome of the cell.

2. The method according to claim 1, wherein at least three double-stranded polynucleotides are introduced into the cell,
wherein a part of the first of the at least three double-stranded polynucleotides has sequence identity with a part of the second of the at least three double-stranded polynucleotides and wherein a part of the second of the at least three double-stranded polynucleotides has sequence identity with the third of the at least three double-stranded polynucleotides, such that, within the cell, the at least three double-stranded polynucleotides can assemble into a double-stranded polynucleotide construct,
wherein the double-stranded polynucleotide construct has at a 5′-end thereof sequence identity with the genome of the cell within proximity of a break in the genome of the cell and wherein the double-stranded polynucleotide construct has at a 3′-end thereof sequence identity with the genome of the cell within proximity of a break in the genome of the cell, and
wherein the double-stranded polynucleotide construct integrates into the genome of the cell within proximity of a break in the genome of the cell.

3. The method according to claim 2, wherein at least four, optionally five, six, seven, eight, nine, ten, eleven, twelve, thirteen, fourteen, fifteen, sixteen, seventeen or eighteen double-stranded polynucleotides are introduced into the cell that are capable of assembly into the double-stranded polynucleotide construct.

4. The method according to claim 1, wherein the double-stranded polynucleotide construct comprises at least one, optionally two, three, four, five, six, seven or eight coding sequences.

5. The method according to claim 1, wherein a plurality of double-stranded polynucleotides is introduced into the cell and assembles in a library of distinct double-stranded polynucleotide constructs, wherein each double-stranded polynucleotide construct is assembled from at least two double-stranded polynucleotides.

6. The method according to claim 1, wherein the cell is an eukaryotic cell, optionally a fungal cell, optionally a non-conventional yeast cell, optionally a Yarrowia cell, optionally a Yarrowia lipolytica cell and wherein the eukaryotic cell optionally is deficient in an NHEJ (non-homologous end joining) component.

7. The method according to claim 1, wherein the break is induced by a functional genome editing system, optionally TALENs, CRISPR/Cas, CRISPR/Cpf1, or I-Scel.

8. The method according to claim 1, wherein the cell expresses a functional heterologous genome editing enzyme, optionally a Cas enzyme, optionally Cas9 or Cas9 nickase; Cpf1; I-Scel, or wherein in the cell a heterologous genome editing enzyme, optionally a Cas enzyme, optionally Cas9 or Cas9 nickase; Cpf1; I-Scel, is present.

9. A composition comprising the cell and at least one of at least two double-stranded polynucleotides as defined in claim 1 and further comprising a vector, optionally a plasmid.

10. A cell comprising an assembled double-stranded polynucleotide construct, obtainable by the method according to claim 1.

11. A cell obtainable by or produced by a method according to claim 1, or a cell comprising an assembled double-stranded polynucleotide construct, obtainable by the method, further comprising a polynucleotide encoding a compound of interest.

12. The cell according to claim 11, expressing the compound of interest.

13. The cell according to claim 11, wherein the compound of interest is foreign to the cell.

14. A method for production of a compound of interest, comprising culturing the cell according to claim 11 under conditions conducive to the production of the compound of interest, and, optionally, purifying or isolating the compound of interest.

15. A product comprising a plurality of double-stranded polynucleotides in genome editing, wherein one or more parts of members of the plurality of double-stranded polynucleotides have sequence identity with one or more parts of other members such that said part can, within a cell, assemble into a double-stranded polynucleotide construct and wherein the double-stranded polynucleotide construct(s) can integrate into a genome of the cell within a proximity of a break into the genome.
